SEQUENCE LISTING (36 sequences)

SEQ ID NO: 1 - DNA, 55 bp, Artificial sequence
  Oligonucleotide used to make a polyhedron
acattcctaagtctgaaacattacagcttgctacacgagaagagccgccatagta

SEQ ID NO: 2 - DNA, 55 bp, Artificial sequence
  Oligonucleotide used to make a polyhedron
tatcaccaggcagttgacagtgtagcaagctgtaatagatgcgagggtccaatac

SEQ ID NO: 3 - DNA, 55 bp, Artificial sequence
  Oligonucleotide used to make a polyhedron
tcaactgcctggtgataaaacgacactacgtgggaatctactatggcggctcttc

SEQ ID NO: 4 - DNA, 55 bp, Artificial sequence
  Oligonucleotide used to make a polyhedron
ttcagacttaggaatgtgcttcccacgtagtgtcgtttgtattggaccctcgcat

SEQ ID NO: 5 - DNA, 55 bp, Artificial sequence
  Oligonucleotide used to make a polyhedron
acattcctaagtctgaaacttcccacgtagtgtcgtttgtattggaccctcgcat

SEQ ID NO: 6 - DNA, 55 bp, Artificial sequence
  Oligonucleotide used to make a polyhedron
ttcagacttaggaatgtgcattacagcttgctacacgagaagagccgccatagta

SEQ ID NO: 7 - DNA, 63 bp, Artificial sequence
  Oligonucleotide used to make a polyhedron
aggcagttgagacgaacattcctaagtctgaaatttatcacccgccatagtagacgtatcacc

SEQ ID NO: 8 - DNA, 63 bp, Artificial sequence
  Oligonucleotide used to make a polyhedron
cttgctacacgattcagacttaggaatgttcgacatgcgagggtccaataccgacgattacag

SEQ ID NO: 9 - DNA, 63 bp, Artificial sequence
  Oligonucleotide used to make a polyhedron
ggtgataaaacgtgtagcaagctgtaatcgacgggaagagcatgcccatccactactatggcg

SEQ ID NO: 10 - DNA, 63 bp, Artificial sequence
  Oligonucleotide used to make a polyhedron
ctgcgcggatgactcaactgcctggtgatacgatctagtctctacgtcaagtaagaaccttag

SEQ ID NO: 11 - DNA, 63 bp, Artificial sequence
  Oligonucleotide used to make a polyhedron
cctcgcatgacatccgcgcagctaaggttcaaagttcctgccgcttcacggacggtattggac

SEQ ID NO: 12 - DNA, 63 bp, Artificial sequence
  Oligonucleotide used to make a polyhedron
ctcttcccgaccgtgaagcggcaggaacttatacttgacgtagagactagaaggatgggcatg

SEQ ID NO: 13 - DNA, 63 bp, Artificial sequence
  Oligonucleotide used to make a polyhedron
ctaagtctgaaatttatcacccgccatagtagacgtatcaccaggcagttgagacgaacattc

SEQ ID NO: 14 - DNA, 63 bp, Artificial sequence
  Oligonucleotide used to make a polyhedron
ctggtgatacgatctagtctctacgtcaagtaagaaccttagctgcgcggatgactcaactgc

SEQ ID NO: 15 - DNA, 63 bp, Artificial sequence
  Oligonucleotide used to make a polyhedron
gcttcacggacggtattggaccctcgcatgacatccgcgcagctaaggttcaaagttcctgcc

SEQ ID NO: 16 - DNA, 63 bp, Artificial sequence
  Oligonucleotide used to make a polyhedron
gagactagaaggatgggcatgctcttcccgaccgtgaagcggcaggaacttatacttgacgta

SEQ ID NO: 17 - DNA, 63 bp, Artificial sequence
  Oligonucleotide used to make a polyhedron
cctcgcatgactcaactgcctggtgatacgaggatgggcatgctcttcccgacggtattggac

SEQ ID NO: 18 - DNA, 62 bp, Artificial sequence
  Oligonucleotide used to make a polyhedron
tcaccaggcagttgagacgaacattcctaagtctgaaatttatcacccgccatagtagacgt

SEQ ID NO: 19 - DNA, 63 bp, Artificial sequence; identical to SEQ ID NO: 8
  Oligonucleotide used to make a polyhedron

SEQ ID NO: 20 - DNA, 63 bp, Artificial sequence; identical to SEQ ID NO: 9
  Oligonucleotide used to make a polyhedron

SEQ ID NO: 21 - DNA, 63 bp, Artificial sequence; identical to SEQ ID NO: 17
  Oligonucleotide used to make a polyhedron

SEQ ID NO: 22 - DNA, 63 bp, Artificial sequence; identical to SEQ ID NO: 7
  Oligonucleotide used to make a polyhedron

SEQ ID NO: 23 - DNA, 63 bp, Artificial sequence; identical to SEQ ID NO: 8
  Oligonucleotide used to make a polyhedron

SEQ ID NO: 24 - DNA, 73 bp, Artificial sequence
  Oligonucleotide used to make a polyhedron
ggtgataaaacgtgtagcaagctgtaatcgactctagcggaagaacccacaaccgcggctcactactatggcg

SEQ ID NO: 25 - DNA, 53 bp, Artificial sequence
  Oligonucleotide used to make a polyhedron
tagagacggtattggaccctcgcatgactcaactgcctggtgatacgagagcc

SEQ ID NO: 26 - DNA, 53 bp, Artificial sequence; identical to SEQ ID NO: 25
  Oligonucleotide used to make a polyhedron

SEQ ID NO: 27 - DNA, 30 bp, Artificial sequence
  Oligonucleotide used to make a polyhedron
gcggttgtgggttcttccgccttcctctcg

SEQ ID NO: 28 - DNA, 30 bp, Artificial sequence
  Oligonucleotide used to make a polyhedron
cgagaggaaggcggaagaacccacaaccgc

SEQ ID NO: 29 - DNA, 53 bp, Artificial sequence
  Oligonucleotide used to make a polyhedron
ctgaaatttatcacccgccatagtagacgtatcaccaggcagttgagacgaac

SEQ ID NO: 30 - DNA, 73 bp, Artificial sequence
  Oligonucleotide used to make a polyhedron
cttgctacacgattcagggcgcgagacctacagcgccgttcgacatgcgagggtccaataccgacgattacag

SEQ ID NO: 31 - DNA, 73 bp, Artificial sequence; identical to SEQ ID NO: 24
  Oligonucleotide used to make a polyhedron

SEQ ID NO: 32 - DNA, 53 bp, Artificial sequence; identical to SEQ ID NO: 25
  Oligonucleotide used to make a polyhedron

SEQ ID NO: 33 - DNA, 30 bp, Artificial sequence; identical to SEQ ID NO: 27
  Oligonucleotide used to make a polyhedron

SEQ ID NO: 34 - DNA, 30 bp, Artificial sequence; identical to SEQ ID NO: 28
  Oligonucleotide used to make a polyhedron

SEQ ID NO: 35 - DNA, 30 bp, Artificial sequence
  Oligonucleotide used to make a polyhedron
ggcgttgtaggtctctcgccttcatggact

SEQ ID NO: 36 - DNA, 30 bp, Artificial sequence
  Oligonucleotide used to make a polyhedron
agtccatgaaggcgagagacctacaacgcc

(19) United States
(12) Patent Application Publication
(10) Pub. No.: US 2009/0227774 A1
(43) Pub. Date: Sep. 10, 2009

(54) Polyhedral Nanostructures Formed from Nucleic Acids

(75) Inventors: Andrew Jonathan Turberfield, Oxford (GB); Christoph Erben, Bicester (GB); Russell Goodman, Oxford (GB)

Correspondence Address: CLARK & ELBING LLP, 101 FEDERAL STREET, BOSTON, MA, 02110, US

(73) Assignee: Isis Innovation Limited, Oxford (GB)

(21) Appl. No.: 12/297,762

(22) PCT Filed: Apr. 20, 2007
(86) PCT No.: PCT/GB2007/001457

§ 371 Date: Mar. 5, 2009

(30) Foreign Application Priority Data
Apr. 20, 2006 (GB) 0607866.1

(51) Int. Cl. C07H 21/04; C07K 14/80

(52) U.S. Cl. 530/358; 536/23.1

(57) ABSTRACT

The present invention provides a nucleic acid polyhedron having a moiety associated therewith together with methods of making the nucleic acid polyhedron.

### FIELD OF THE INVENTION

The present invention relates to nanostructures and in particular to polyhedra formed from a nucleic acid. In particular, the present invention provides a nucleic acid polyhedron having a moiety contained therein and methods of making it.

### BACKGROUND TO THE INVENTION

The use of DNA has been investigated in the production of a variety of nanostructures. The 50 nm persistence length (Manning Biopolymers 198120, 1751) and sequence specific base pairing of DNA make it a useful material for the construction of objects on the nanometre length scale (Seeman, Nature 2003 421, 427). Nanostructures that have been made include simple machines (Yurke et al Nature 2000 406, 605 and Yan et al Nature 2002 415, 6867), extended periodic arrays (Winfree et al Nature 1998 394, 539) and discrete three-dimensional nanostructures (Chen et al Nature 1991 350, 631 and Zhang et al J. Am Chem Soc 1994 116, 1661).

A polyhedron is a three dimensional shape that is made up of a finite number of polygonal faces. A nucleic acid polyhedron is a polyhedron in which strands of a nucleic acid, such as DNA, form at least some of or part of the edges where the faces meet. The edges of the polyhedron meet at vertices. DNA polyhedra that have been produced include a cube, a truncated octahedron using a solid support strategy that relies on repeated enzymatic treatments and purification, and a regular octahedron incorporating paranemic crossovers. The single step synthesis of a DNA tetrahedron has also been described (Goodman et al Chem. Comun. 2004 12, 1372-1373 and Goodman et al Science 2005 310, 1661-1665).

### SUMMARY OF THE PRESENT INVENTION

The present inventors have shown that a nucleic acid polyhedron containing a moiety can be synthesized. Accordingly, the invention provides a nucleic acid polyhedron having a moiety contained therein. The invention also provides a method of making a nucleic acid polyhedron having a moiety contained therein, comprising:

(a) providing one or more oligonucleotides, each oligonucleotide comprising subsequences that hybridize to subsequences on the same oligonucleotide or a different oligonucleotide to allow the oligonucleotide(s) to form the polyhedron;

(b) mixing the oligonucleotide(s) of (a) and the moiety in a buffer solution; and

(c) optionally subjecting the mixture of (b) to a heat treatment comprising a heating step to denature the oligonucleotide(s) and a cooling step to anneal the oligonucleotide(s) to allow them to hybridize to form thereby a nucleic acid polyhedron;

wherein step (b) and step (c) if it is performed are carried out in such a manner that the moiety becomes contained within the nucleic acid polyhedron.

The invention further provides a nucleic acid polyhedron having a moiety attached to the outside of the polyhedron.

### DESCRIPTION OF THE FIGURES

FIG. 1. (a) Synthetic scheme for the DNA tetrahedron, with Schlegel representations of the final product illustrating both possible diasteromers. The products of edge digestions that cut the central (E, F) or end (A, B, C, D) subsequences are also illustrated. (b) Two different views of a space filling representation of a DNA tetrahedron with 17 bp edges and 2 bp hinges.

FIG. 2. (a) Synthesis of the DNA tetrahedron. Lane 1: 50 bp ladder. Lane 2: strands 1+2. Lane 3: strands 3+4. Lane 4: strands 1+2+3. Lane 5: strands 1+2+4. Lane 6: strands 1+3+4. Lane 7: strands 2+3+4. Lane 8: strands 1+2+3+4. (b) Edgewise digest of tetrahedron. Lane 1: edge A digest. Lane 2: uncut tetrahedron. Lane 3: edge B digest. Lane 4: edge C digest. Lane 5: edge D digest. Lane 6: edge E digest. Lane 7: edge F digest.

FIG. 3. (a) Synthetic scheme for the creation of dimeric constructs. The intermediates when combined form the dimer. The strands joining the two intermediates share a restriction site for the enzyme Dde I, and when exposed to this enzyme form products similar to an edge digestion of the tetrahedron. (b) Lane 1: tetrahedron. Lane 2: intermediate 1. Lane 3: intermediate 2. Lane 4: intermediate 1+2. Lane 5: intermediate 1+2 heated to 95° C. and cooled to room temperature. Lane 6: Dde I digest of lane 4 products. Lane 7: Dde I digest of lane 1 products.

FIG. 4. (A) Two different views of a model of a regular DNA octahedron. (B) A Schlegel representation showing the specific sequences involved in the construction of a particular DNA octahedron. The nicks are indicated, and each edge, formed by the hybridization of two oligonucleotides, is marked uniquely. (C) Gel evidence of the successful construction of a DNA octahedron from eight 63 nt oligonucleotides. In this denaturing gel, hybridization interactions are eliminated so that the component oligonucleotides, unless circularized and concatenated, migrate independently. Control lanes C1 and C2 contain linear and circular 63 nt oligonucleotides. Lane 1 contains a gel-purified DNA octahedron with no nicks ligated: as expected, only linear DNA products are obtained. Lanes 2-9 contain 8 different gel-purified octahedra; in each case a single oligonucleotide (different in each case) has been ligated, and both linear and circular DNA products are produced. This result demonstrates that the purified octahedron contains each of the eight component oligonucleotides, and that during the ligation step (before denaturation) the 5′ and 3′ ends of each are held adjacent to each other by hybridization to a common splint, consistent with the designed structure.

FIG. 5. (a) Molecular model of a DNA trigonal bipyramid and (b) positions of the six constituent DNA 63-mers (a-c and A-C).

FIG. 6. Analysis of the bipyramid by (a) native and (b) denaturing polyacrylamide gel electrophoresis (PAGE). Lane 1: unpurified products of bipyramid synthesis. Lane 1′: (control) strand a. Subsequent lanes in both native and denaturing gels contain gel-purified tetrahedra (controls) and bipyramids with one or two of the constituent strands ligated (ligated strands are identified above each lane). Schematics indicate relationships between faces around which ligated strands are designed to run.

FIG. 7. Denaturing PAGE analysis of bipyramids with more than two phosphorylated strands. Control lanes contain DNA tetrahedra in which the pattern of linkage between strands is known. All samples were incubated with T4 DNA ligase and purified from native polyacrylamide gels. The number of phosphate modifications and hence ligated nicks is given above the lane for each sample. Dashed vertical lines separate samples with the same number of linked circles but different patterns of linkage. Phosphorylated strands in the bipyramid samples are: lane 4: A, B, C; lane 5: a, b, c; lane 6: a, c, A; lane 7: b, B, C; lane 10: b, c, A, C; lane 11: a, c, A, B; lane 12: b, A, B, C; lane 13: a, b, c, C; lane 14: a, b, A, B; lane 15: a, c, A, C; lane 16: all but B; lane 17: all but c; lane 18 and lane 22: all; lane 23: all but B; lane 24: all but A; lane 25: all but C; lane 26: all but c; lane 27: all but b; lane 28: all but a. The denaturing PAGE gels have 5% (29:1) stacking layers and 12% (29:1), 8% (19:1) and 6% (19:1) separating layers respectively from left to right.

FIG. 8. Formation of bipyramids using different nick positions. The PAGE gel shows the products of formation corresponding to six different bipyramid designs. The base sequences of each edge are identical; only nick positions differ. For all six patterns of nick placement there is significant yield of the bipyramid product, although formation efficiencies differ. The strands used were as follows: Sample 1: a, b, c, A, B, C; Sample 2: a*, b, c, A, B* C*; Sample 3: a*, b, c, A*, B, C; Sample 4: a*, b, c, A*, B*, C*; Sample 5: a, b, c, A, B*, C; Sample 6: a*, b, c, A*, B*, C; and Control lane: 50 base pair DNA ladder.

FIG. 9. Formation of bipyramids using different annealing protocols. Samples were held at the indicated temperature for 3 minutes followed by fast cooling to 4° C. Lane 94°ₛ: the sample was heated to 94° C. and cooled to room temperature over ˜15 minutes.

FIG. 10. Native PAGE gels showing bipyramids with 5 or 6 nicks ligated after incubation with and without exonuclease III (Exo III). The bipyramid band survives the enzymatic digestion only if all six nicks have been ligated; this demonstrates that the product identified as the bipyramid does contain all six strands of DNA, as designed.

FIG. 11. Positioning a protein within a DNA tetrahedron. a) Tetrahedron design: the black arrow indicates the protein attachment site at the 5′ end of s1. b) Molecular model showing cytochrome c inside the tetrahedron. c) Native polyacrylamide gel showing the variation in electrophoretic mobility with the position of the attachment site. Control T: unmodified tetrahedron.

FIG. 12. Confirmation of the identity of the s1-protein conjugate. Left: Native gel of samples after incubation with Proteinase K. Right: SDS denaturing gel after incubation with Exonuclease I. Only the band that we have identified as the conjugate is digested by both enzymes, as expected.

FIG. 13. Analysis of protein-tetrahedron complexes. a) Native gel showing constructs with cytochrome c attached at the 8^{th} and 13^{th} bases of one edge (inside and outside the cage, respectively). Bands A-F are purified from the indicated regions of similar gels. b) Denaturing gel to analyze contents of bands A-F. Controls c8, c13: cytochrome c conjugated to oligonucleotides s1.

FIG. 14. Denaturing PAGE analysis of the contents of all bands in FIG. 4c after ligation and gel purification for all attachment positions. Controls: s1: oligonucleotide s1; c: s1-cytochrome c conjugate; T(4L): unmodified tetrahedron with all four oligonucleotides s1-s4 ligated; T(3L): like T(4L), but s1 unligated. All lanes contain partially disassembled tetrahedra and the oligonucleotide-protein conjugate, as expected.

FIG. 15. Schematic representation of a design of cross-bridges. (a) A normal DNA tetrahedron formed from 4 different oligonucleotides. (b) A tetrahedron with a cross-bridge across one face. The cross-bridge is formed by connecting the 3′ end of one strand (left-hand edge) and the 5′ end of another strand (right-hand edge) by hybridization with a splint strand. This cross-bridge is formed by using longer versions of the normal tetrahedra oligos with overhangs on the 5′ and/or 3′ end.

FIG. 16. PAGE Gel. Left Lane: DNA tetrahedra. Other lanes from left to right: DNA tetrahedra with cross-bridges with a length of 16 to 19 bases, with and without the splint strand bound to the cross-bridge.

FIG. 17. Synthetic scheme illustrating how the device works (A), and 3D models of two different views of the open and closed version of the tetrahedron (B).

FIG. 18. A: A tetrahedron with a single reconfigurable arm forms in high yield from the four component strands. B: Denaturing gel analysis of the tetrahedron confirms the expected topology.

FIG. 19. A: Gel analysis of repeated opening and closing of the tetrahedron. B: Bulk FRET analysis of the repeated opening and closing.

FIG. 20. Gel analysis of various controls confirming that the DNA tetrahedron with the hairpin is actually ‘closed’ at room temp (i.e. the hairpin is formed as expected).

FIG. 21. Schematic of how two dynamic motifs can be incorporated into different edges of a single tetrahedron. All four possible states are illustrated.

FIG. 22. Gel analysis of all four possible states of a tetrahedron with two edges modified to incorporate dynamic motif.

FIG. 23. (a) Synthesis scheme for a DNA tetrahedron with a single reconfigurable edge. Four strands are combined in solution to form a tetrahedron with five 20 bp edges, and one 10 bp edge containing a hairpin loop. This edge may be extended or contracted by the addition of the appropriate DNA strands. (b) Two different views of the open and closed states of tetrahedron described in (a). The closed state, illustrated with the two sections of the hairpin-containing arm coaxially stacked, can be extended in length from approximately 3.4 nm to 10.2 nm.

FIG. 24. Assembly and denaturing gel analysis of a DNA tetrahedron with a single reconfigurable edge. (a) Native gel analysis of the assembly. When strands 1 (63 nt, lane 1), 2 (63 nt, lane 2), 3 (73 nt, lane 3), and 4 (53 nt, lane 4) are combined together in equal quantities, a high molecular weight band in addition to some low molecular weight products is produced (lane 5). Ligation with T4 DNA ligase results in single-band product in high yield (lane 6) which can be gel purified (lane 7) if desired. Note that strand 4 is not 5′-phosphorylated. (b) Denaturing gel analysis of the same gel-purified, ligated, tetrahedron. Lanes 1-3 provide controls of strands of length 53 nt (lane 1), 63 nt (lane 2), and 73 nt (lane 3). A gel-purified version of the tetrahedron which has not been ligated is resolved into these component strands (lane 4). When ligated and treated with exonuclease III, the tetrahedron is resolved into several different circular catenanes of the appropriate mobility. The box illustrates an area of the gel where differential contrast has been applied to display the 63 nt circular catenane. The 73 nt circular catenane is not visible, but is present in the higher order dual and triple catenanes. Lane 6 is a control of a ligated, gel-purified regular 20 bp tetrahedron treated with exonuclease III to remove linear DNA strands, and, as expected, shares several bands with the reconfigurable tetrahedron.

FIG. 25. Cycling between the open and closed state of a tetrahedron with a single reconfigurable edge. (a) Native electrophoresis analysis of state switching. 9% 19:1 1×TAE PAGE. This figure shows the change in mobility between the closed state of the tetrahedron (lanes 1, 3, and 5) and its open state (lanes 2, 4, and 6). Successive additions of fuel and antifuel strands produces waste duplex as well as excess hairpin strands. (b) Bulk FRET measurement of a tetrahedron containing CY3/CY5 modifications at the 5′ and 3′ of the nick opposite the hairpin loop. Successive additions of fuel (black arrows) and antifuel strands (dashed arrows) produce the expected changes in fluorescent intensity. (c) Single molecule FRET measurements of the initial conversion of the closed tetrahedron (left panel) to the open tetrahedron (right panel) indicates a single population of fluorescent objects in each case. E is the donor-accept FRET efficiency, and S is the donor-accept stoichiometry factor. The data have been filtered to remove molecules containing either only donor or acceptor fluorophores.

FIG. 26. Assembly and denaturing gel analysis of a DNA tetrahedron with a two independently reconfigurable, opposite edges (see Supplementary Data 1 for specific sequences). (a) Native gel analysis of assembly. When strands 1 (53 nt, lane 1), 2 (73 nt, lane 2), 3 (73 nt, lane 3), 4 (53 nt, lane 4) are combined in equal quantities, a high molecular weight band is produced. When treated with T4 DNA ligase (lane 6), a slight shift in mobility is observed. Not that strands 1 and 4 are not 5′-phosphorylated. The band can be gel purified (lane 7). (b) Denaturing gel analysis of the same tetrahedron. Lanes 1 and 2 provide controls for strands of 53 nt (lane 1) and 73 nt lengths. An unligated tetrahedron is resolved into strands of these lengths (lane 3). When ligated and treated with exonuclease III, two circular catenanes are produced, corresponding to a 83 nt single circle and a dual 83/83 nt catenane (lane 4). Lane 5 is a control of a regular 20 bp, ligated tetrahedron treated with exonuclease III.

FIG. 27. State switching in a tetrahedron with two opposite reconfigurable edges, each independently addressable. 9% 19:1 PAGE 1×TAE 15 mM MgCl₂. This figure illustrates how every state may be reached in a single step from every other possible state. For example, to get from a completed closed state (lanes 1 and 12) to a completely open state, both fuels may be added simultaneously (lane 6). They may be closed separately (lanes 7 and 8), or simultaneously (lane 9) by the appropriate anti-fuel additions.

FIG. 28. Control experiments to verify hairpin loop closure in experimental conditions. (a) This figure illustrates schematics of seven different versions of a tetrahedron with a single reconfigurable edge. Version I-III differ in the length of the hairpin loop and neck regions; in each case, the total hairpin length is 20 nt. Version IV possesses no loop secondary structure. Versions V-VII contain identical hairpin loop structures to versions I-III, but differ in the placement of the nick opposite the hairpin loop: in these cases, the nick has been moved to an adjacent strand. This is expected to increased the probability of the formation of a stable hairpin loop. (b) Gel analysis of controls of (a) in a gel containing 15 mM MgCl₂. 9% 19:1 1×TAE 15 mM MgCl₂. Lane 1: hairpin-loop hybridized to fuel strand. Lane 2: hairpin-loop with no secondary structure (IV). Lanes 3, 4, 6, 8: hairpin-loops with necks of 4(I), 4(I), 5(II), and 6(III) bp. Lanes 5, 7, and 9: hairpin-loops with necks of 4(V), 5(VI), and 6(VII) bp without a nick. The sequence of the hairpin in lane 3 corresponds to that of the hairpin in FIG. 25, whereas that in lane 3 corresponds to the additional hairpin of the reconfigurable tetrahedron described in FIG. 27. Note that while the tetrahedron hybridized to fuel and the tetrahedron with no secondary structures have distinct mobilities, no difference in the mobilities of tetrahedron with hairpins of different lengths (nicked or unnicked) are evident. This is taken as proof that the hairpins used in this paper are properly closed in the conditions used. In ionic strengths less than 15 mM MgCl₂, this is not true (data not shown).

### DETAILED DESCRIPTION OF THE INVENTION

The present inventors have shown for the first time that a nucleic acid polyhedron containing a moiety can be synthesized in a simple manner. The nucleic acid polyhedron is formed by the hybridization of one or more specially designed oligonucleotides. The present inventors have shown that the presence of the moiety does not interfere with the hybridization of the oligonucleotides such that the nucleic acid polyhedron can form around the moiety. The present invention therefore provides a nucleic acid polyhedron having a moiety contained therein and methods of making it.

### Nucleic Acid Polyhedron

In accordance with the present invention, a nucleic acid polyhedron is a polyhedron in which strands of a nucleic acid form at least part of the edges where the faces meet. It is preferred that a nucleic acid forms all of the edges of the nucleic acid polyhedron. The nucleic acid is preferably DNA. The DNA used to make the polyhedron may be naturally-occurring, modified or synthetic DNA, including RNA-DNA chimeras or oligonucleotides incorporating modified backbones. In some embodiments, the nucleic acid is RNA, PNA or LNA.

The nucleic acid polyhedron can be designed to have any number of faces, edges and/or vertices. For example, the nucleic acid polyhedron can be designed to have 4, 5, 6, 7, 8, 9, 10, 11, 12, 13, 14 or more faces. The nucleic acid polyhedron is preferably a tetrahedron, a cube (hexahedron), a pyramid, a dipyramid or an octahedron. The nucleic acid polyhedron can be regular or irregular. The nucleic acid polyhedron is preferably rigid.

The nucleic acid polyhedron can comprise at least one nucleic acid cross-bridge. The structure of the nucleic acid polyhedron is preferably modified to allow for release of the moiety. These embodiments are discussed in more detail below.

The nucleic acid polyhedron can be selected to have any size. The size of the nucleic acid polyhedron is typically chosen based on the size of the moiety. The edges of the nucleic acid polyhedron are preferably at least 5, at least 10, at least 20 or at least 30 or at least 40 or at least 50 bases pairs in length, such as from 5 to 100, from 10 to 75, from 15 to 50 or from 20 to 30 nucleotides in length.

The nucleic acid polyhedron may be detectably labeled. Suitable labels include, but are not limited to, fluorescent molecules, radioisotopes, e.g. ¹²⁵I, ³⁵S, enzymes, antibodies, polynucleotides and linkers such as biotin.

### Contained Inside

In accordance with the present invention, the moiety is contained inside the nucleic acid polyhedron or encapsulated by the nucleic acid polyhedron. The size of the nucleic acid polyhedron, and in particular the size of the polygonal faces, can be selected so that the moiety is trapped inside the nucleic acid polyhedron in the absence of cleaving or opening the nucleic acid polyhedron. The nucleic acid polyhedron preferably protects the moiety from the factors, such as antibodies or enzymes, outside the polyhedron. For example, the polyhedron can be selected to prevent antibodies or enzymes from contacting the moiety.

In one embodiment, the moiety is attached to or bound to an internal edge or an internal vertex of the nucleic acid polyhedron. The moiety is preferably attached to or bound to the inside of the nucleic acid polyhedron by an ionic interaction, a covalent linkage, hydrogen bonding or van der Waals forces. The moiety may be attached or bound to the inside of the nucleic acid polyhedron via one or more linker molecules. For example, the moiety can be attached to or bound to the inside of the cage using an aptamer or a poly-histidine tag. Alternatively, the moiety can be attached to or bound to the nucleic acid polyhedron using bifunctional cross-linker molecules, such as sulfosuccinimidyl 4-(N-maleimidomethyl)cyclohexane-1-carboxylate (sulfo-SMCC) and SPDP (N-succinimidyl 3-(2-pyridyldithio)propionate).

In another embodiment, the moiety is not attached or bound to the inside of the nucleic acid polyhedron.

### Moiety

In accordance with the present invention, a moiety is contained inside the nucleic acid polyhedron. The nucleic acid polyhedron can have more than one moiety contained within it. The nucleic acid polyhedron can have two or more moieties that are the same or different contained within it. For example, the nucleic acid polyhedron may have 2, 3, 4, 5, 6, 7, 8, 9, 10 or more moieties that are the same or different contained within it.

The moiety can be any size as long as it fits inside the nucleic acid polyhedron. The moiety is preferably less than 100 nm, less than 75 nm, less than 50 nm or less than 25 nm, such as from 1 to 100 nm, from 5 to 100 nm, from 10 to 100 nm, from 25 to 100 nm, from 50 to 100 nm, from 1 to 75 nm, from 5 to 50 nm or from 10 to 25 nm, at its widest part. For example, a substantially spherical moiety preferably has a diameter of less than 100 nm. Typically, a moiety that is from 1 to 100 nm at its widest part is selected to fit into a nucleic acid polyhedron whose edges are of such a length that a notional sphere of a diameter between 1 and 100 nm could be positioned within the polyhedron. Typically, in the case of a regular tetrahedron, the length of the edges is at least 1.4 times the width of the moiety at its widest part. For example, a moiety that is 10 nm at its widest part is selected to fit into a regular tetrahedron whose edges are at least 14 nm in length and a moiety that is 100 nm at its widest part is selected to fit into a regular tetrahedron whose edges are at least 140 nm in length.

The moiety can be any substance or collection of substances. The moiety can be an inorganic compound, such as a metal, metal complex or mineral, an organic compound or a polymer. For example, the moiety can be a semiconductor nanocrystal (quantum dot). The moiety is preferably selected to have a biological effect. For example, the moiety can be a therapeutic agent or an enzyme cofactor. The moiety can also be a diagnostic agent. In some embodiments, the moiety is not a nucleic acid moeity.

The moiety is preferably a peptide, polypeptide or a protein. The peptide, polypeptide or protein may be naturally-occurring or non-naturally-occurring. The polypeptide or protein may include within it synthetic or modified amino acids. A number of different types of modification to amino acids are known in the art. For the purposes of the invention, it is to be understood that the peptide, polypeptide or protein described herein may be modified by any method available in the art.

Examples of a moiety include an antibody, an enzyme, a hormone, a growth factor, a growth regulatory protein, a cytokine, a bacterial protein, a virus particle, a virus protein and a parasite-derived protein. The cytokine may be selected from an interleukin, preferably IFN-1, IL-2, IL-4, IL-5, IL-6, IL-10, IL-12 or IL-13, an interferon, preferably IL-γ or other cytokines such as TNF-α. The moiety can be cytochrome c.

In some embodiments, the moiety is a nucleic acid, such as DNA or RNA. The nucleic acid can be single stranded or double stranded. In some embodiments, the moiety is not a single stranded nucleic acid. The nucleic acid can be any length provided that it can be substantially enclosed within the nucleic acid polyhedron. The nucleic acid is preferably greater than 11 nucleotides in length, such as greater than 21 nucleotides in length. The nucleic acid is preferably greater than 7 nm in length, such as greater than 14 nm in length. The nucleic acid is preferably selected to encode a protein. The nucleic acid can be selected to encode any of the proteins described above. Examples of a moiety include a DNA oligonucleotide, small interfering RNA (siRNA) or micro-RNA (miRNA).

The moiety may be detectably labeled. Suitable labels include, but are not limited to, fluorescent molecules, radioisotopes, e.g. ¹²⁵I, ³⁵S, enzymes, antibodies, polynucleotides and linkers such as biotin.

### Making a Nucleic Acid Polyhedron Having a Moiety Contained Therein

At least part of the nucleic acid polyhedron is a double stranded nucleic acid. Part of each edge of the nucleic acid polyhedron is preferably a double stranded nucleic acid. Most of each edge of the nucleic acid polyhedron is more preferably a double stranded nucleic acid. For example, each edge can include at most 1, 2, 3, 4, 5, 10, 15 or 20 bases of a single stranded nucleic acid. All of each edge can be a double stranded nucleic acid.

The nucleic acid polyhedron is made from one or more oligonucleotides. The double stranded nucleic acid that makes up all or part of each edge is formed from the hybridization of an oligonucleotide to itself or by the hybridization of one oligonucleotide to another oligonucleotide. If one oligonucleotide is used to make the nucleic acid polyhedron, the oligonucleotide is selected so that it hybridizes to itself. If more than one oligonucleotide is used to make the nucleic acid polyhedron, the oligonucleotides are selected so that they hybridize to themselves and/or each other. Thus an oligonucleotide comprises one or more subsequences, each of the subsequences being designed so that they can hybridize to subsequences on the same oligonucleotide or a different oligonucleotide. In one embodiment, the subsequences are linked by linkers. In another embodiment, the subsequences are not linked by linkers. The oligonucleotides are preferably selected to allow self-assembly of the nucleic acid polyhedron.

The oligonucleotides can be designed to run around an entire face or entire faces of the nucleic acid polyhedron. The oligonucleotides can also be designed to run around part of one or more of the faces of the nucleic acid polyhedron. The oligonucleotides are preferably designed such that the two strands that form all or part of an edge separate at or near one of the vertices such that at least one of the two strands forms at least a part of one of the other edges that meet at the vertex. The oligonucleotides are more preferably designed such that the two strands that form all or part of an edge separate at or near one of the vertices such that each of the two strands forms part of different edges that meet at the vertex. Separation of the two strands such that each of the strands forms part of different edges restricts rotation of the edge formed from the two strands around its axis. The polyhedron can be designed such that separation of the two strands forming part or all of an edge occurs at one or more of the vertices of the nucleic acid polyhedron, for example at 2, 3, 4, 5, 6, 7, 8 or all of the vertices.

As can be appreciated, the number of oligonucleotides needed to make a nucleic acid polyhedron can vary. For instance, a nucleic acid polyhedron can be made from a single oligonucleotide. Alternatively, a nucleic acid polyhedron can be made from two or more oligonucleotides, such as 3, 4, 5, 6, 7 or 8 or more oligonucleotides. In one specific embodiment, the number of oligonucleotides used to make the nucleic acid polyhedron is equal to the number of faces of the nucleic acid polyhedron. For example, a tetrahedron can be formed from four oligonucleotides. This embodiment is discussed in more detail below. In another embodiment, the number of oligonucleotides used to make the nucleic acid polyhedron is less than the number of faces of the nucleic acid polyhedron. For example, a tetrahedron can be formed from 3 oligonucleotides, such as by covalently linking, by ligation or in synthesis, two oligonucleotides, each of which runs around one face of the polyhedron, through an inserted polynucleotide linker. The linker may cross one face or between faces to form a cross-bridge or internal division: the formation of cross-bridges is discussed in more detail below. In another embodiment, the number of oligonucleotides used to make the nucleic acid polyhedron is greater than the number of faces of the nucleic acid polyhedron. Short oligonucleotides can be used to hybridize to two or more oligonucleotides that form part of two or more of the edges of the nucleic acid polyhedron in order to create cross-bridges. The formation of cross-bridges is discussed in more detail below. Alternatively, extra nicks can be introduced into a polyhedron so that it is formed from more oligonucleotides than there are faces of the nucleic acid polyhedron. For example, a tetrahedron can be made of 6 oligonucleotides, only 2 of which run round a complete face.

Each oligonucleotide comprises one, two or more subsequences optionally having linkers there between. The linkers are designed and selected such that hybridization of subsequences of each oligonucleotide forms the nucleic acid polyhedron. Thus, in some cases, the linker acts as a hinge region and facilitates the formation of a vertex at which the edges which are connected by the linker meet at an angle which is substantially different from 0° or 180°.

The linkers preferably comprise nucleotides. The nucleotides forming the linkers between subsequences are selected so that no base pairing with the other oligonucleotides making up the polyhedron occurs. In the polyhedron therefore, the linkers form single stranded regions between the hybridized subsequences forming the edges of the polyhedron. The linkers can be of any desired selected length depending on the size of the polyhedron. For example, the length of the linker is preferably at least 1, at least 5, at least 10, at least 20, at least 30 or at least 50 nucleotides, such as from 1 to 100, from 5 to 75, from 10 to 50 or from 15 to 30 nucleotides. Shorter linkers are in general used to provide a more rigid polyhedron.

The oligonucleotides can be of any suitable length to prepare the polyhedron. Each of the oligonucleotides preferably has a length of at least 30, at least 40, at least 50, at least 75, at least 100, at least 200, at least 300, at least 400 or at least 500 nucleotides, such as from 30 to 500, 40 to 450 or from 50 to 400 nucleotides. The lengths of the subsequences are selected depending on the desired size and shape of the polyhedron. The subsequences are preferably at least 5, at least 10, at least 20, at least 30, at least 50, at least 100, at least 200 or at least 300 nucleotides in length, such as from 5 to 300, from 10 to 200, from 15 to 150 or from 20 to 100 nucleotides in length. Where two or more subsequences are used to form a single edge of the polyhedron face, the length of such subsequences is selected to provide a desired total length for the edge. The lengths of the subsequences can be the same or different.

The subsequences are selected such that each subsequence hybridizes to another subsequence on the same oligonucleotide or a different oligonucleotide. The subsequences can be selected to be completely complementary to the other subsequence on the same oligonucleotide or a different oligonucleotide. Alternatively, the subsequences can be selected to include 1, 2 or 5 or more mismatches depending on the length of the subsequences. Mismatches are tolerated as long as the subsequences hybridize to other subsequences to form a nucleic acid polyhedron.

The oligonucleotides may be produced by any suitable method, such as synthetic methods well-known in the art.

The subsequences are formed from a nucleic acid. The subsequences preferably comprise DNA. Synthetic DNA analogues such as RNA-DNA chimeras or oligonucleotides incorporating modified backbones can also be used. The subsequences can also comprise RNA, PNA or LNA. References to a nucleic acid polyhedron used herein also relate to a polyhedron produced using synthetic or modified nucleic acids

In one specific embodiment, the number of oligonucleotides used to form the nucleic acid polyhedron is equal to the number of faces of the polyhedron. Such a nucleic acid polyhedron can be synthesized in accordance with the methods disclosed in Goodman et al Chem. Comun. 2004 12, 1372-1373 and Goodman et al Science 2005 310, 1661-1665. The nucleic acid polyhedron is made from several different oligonucleotides and each oligonucleotide runs around one of the faces of the polyhedron. The oligonucleotides are selected so that the portions of oligonucleotide at the shared edges of the adjacent faces hybridize to each other. Thus, each oligonucleotide comprises two or more subsequences, each of the subsequences being designed so that they can hybridize to subsequences of the oligonucleotides along the shared edge of the adjacent faces. The subsequences are linked by linkers which form hinges which may allow each oligonucleotide to run around each face of the polyhedron.

Each nucleic acid polyhedron is made up of several different oligonucleotides. The number of oligonucleotides will depend on the number of sides that the polyhedron possesses. For example, four oligonucleotides will be used to make a four-sided tetrahedron, six oligonucleotides will be used to make an six-sided cube and eight oligonucleotides will be used to make an eight-sided octahedron.

Each oligonucleotide can comprise three or more subsequences having linkers there between. In one way of making a tetrahedron, each first subsequence is capable of hybridizing to the first subsequence of one of the other oligonucleotides, each second subsequence is capable of hybridizing to the second subsequence of another of the three oligonucleotides and each third subsequence is capable of hybridizing to the third subsequence of the third of the other three oligonucleotides. In another way of making a tetrahedron, the oligonucleotide can comprise four subsequences having linkers therebetween, the first and fourth subsequences effectively making up one edge of one tetrahedron face, the second subsequence and third subsequence making up the other two edges.

Thus, one way of making a nucleic acid tetrahedron involves the use of four oligonucleotides in which

the first sequence of the first oligonucleotide is capable of hybridizing to the first subsequence of the third oligonucleotide;

the second subsequence of the first oligonucleotide is capable of hybridizing to the second subsequence of the second oligonucleotide;

the third subsequence of the first oligonucleotide is capable of hybridizing to the third subsequence of the fourth oligonucleotide;

the first subsequence of the second oligonucleotide is capable of hybridizing to the first subsequence of the fourth oligonucleotide;

the third subsequence of the second oligonucleotide is capable of hybridizing to the third subsequence of the third oligonucleotide; and

the second subsequence of the third oligonucleotide is capable of hybridizing to the second subsequence of the fourth oligonucleotide.

The four oligonucleotides in a tetrahedron can be thus represented as having the sequences (a, b, c), (d, b, e), (a, f, e), (d, f, c) wherein the sequences of the same letter (a, b, c, d, e, f) are capable of hybridizing to one another.

In a more preferred embodiment, making a nucleic acid tetrahedron involves the use of four oligonucleotides in which:

the first and fourth subsequence of the first oligonucleotide are capable of hybridizing to the second subsequence of the fourth oligonucleotide;

the first and fourth subsequence of the second oligonucleotide are capable of hybridizing to the second subsequence of the third oligonucleotide;

the first and fourth subsequence of the third oligonucleotide are capable of hybridizing to the third subsequence of the first oligonucleotide;

the first and fourth subsequence of the fourth oligonucleotide are capable of hybridizing to the third subsequence of the second oligonucleotide;

the second subsequence of the first oligonucleotide is capable of hybridizing to the second subsequence of the second oligonucleotide; and

the third subsequence of the third oligonucleotide is capable of hybridizing to the third subsequence of the fourth oligonucleotide.

The four oligonucleotides can be thus represented as having the sequences (a1, b, c, a2), (d1, b, e, d2), (c1, d, f, c2), (e1, a, f, e2) wherein the sequences of the same letter (a, b, c, d, e, f) are capable of hybridizing to one another.

In one way of making an octahedron, the oligonucleotide can comprise four subsequences having linkers therebetween, the first and fourth subsequences effectively making up one edge of one octahedron face, the second subsequence and third subsequence making up the other two edges.

One way of making a nucleic acid octahedron involves the use of eight oligonucleotides in which:

the first and fourth subsequence of the first oligonucleotide are capable of hybridizing to the second subsequence of the fifth oligonucleotide;

the first and fourth subsequence of the second oligonucleotide are capable of hybridizing to the third subsequence of the third oligonucleotide;

the first and fourth subsequence of the third oligonucleotide are capable of hybridizing to the second subsequence of the seventh oligonucleotide;

the first and fourth subsequence of the fourth oligonucleotide are capable of hybridizing to the third subsequence of the first oligonucleotide;

the first and fourth subsequence of the fifth oligonucleotide are capable of hybridizing to the second subsequence of the sixth oligonucleotide;

the first and fourth subsequence of the sixth oligonucleotide are capable of hybridizing to the third subsequence of the second oligonucleotide;

the first and fourth subsequence of the seventh oligonucleotide are capable of hybridizing to the second subsequence of the eighth oligonucleotide;

the first and fourth subsequence of the eighth oligonucleotide are capable of hybridizing to the third subsequence of the fourth oligonucleotide;

the second subsequence of the first oligonucleotide is capable of hybridizing to the second subsequence of the second oligonucleotide;

the second subsequence of the third oligonucleotide is capable of hybridizing to the second subsequence of the fourth oligonucleotide;

the third subsequence of the fifth oligonucleotide is capable of hybridizing to the third subsequence of the eighth oligonucleotides; and

the third subsequence of the sixth oligonucleotide is capable of hybridizing to the third subsequence of the seventh oligonucleotide

The eight oligonucleotides can be thus represented as having the sequences (a1, b, c, a2), (d1, b, e, d2), (f1, g, d, f2), (c1, g, h, c2), (i1, a, j, i2), (e1, i, k, e2), (l1, f, k, l2), (h1, l, j, h2) wherein the sequences of the same letter (a, b, c, d, e, f, g, h, i, j, k, l) are capable of hybridizing to one another.

In one way of making an bypramid, the oligonucleotide can comprise four subsequences having linkers therebetween, the first and fourth subsequences effectively making up one edge of one trigonal bypramid face, the second subsequence and third subsequence making up the other two edges.

One way of making a nucleic acid bypramid involves the use of six oligonucleotides in which:

the first and fourth subsequence of the first oligonucleotide are capable of hybridizing to the second subsequence of the second oligonucleotide;

the first and fourth subsequence of the second oligonucleotide are capable of hybridizing to the third subsequence of the fifth oligonucleotide;

the first and fourth subsequence of the third oligonucleotide are capable of hybridizing to the third subsequence of the sixth oligonucleotide;

the first and fourth subsequence of the fourth oligonucleotide are capable of hybridizing to the second subsequence of the first oligonucleotide;

the first and fourth subsequence of the fifth oligonucleotide are capable of hybridizing to the second subsequence of the sixth oligonucleotide;

the first and fourth subsequence of the sixth oligonucleotide are capable of hybridizing to the third subsequence of the fourth oligonucleotide;

the third subsequence of the first oligonucleotide is capable of hybridizing to the third subsequence of the third oligonucleotide;

the third subsequence of the second oligonucleotide is capable of hybridizing to the second subsequence of the third oligonucleotide;

the second subsequence of the fourth oligonucleotide is capable of hybridizing to the second subsequence of the fifth oligonucleotides.

The six oligonucleotides can be thus represented as having the sequences (a1, b, c, a2), (d1, a, e, d2), (f1, e, c, f2), (b1, g, h, b2), (i1, g, d, i2), (h1, i, f, h2) wherein the sequences of the same letter (a, b, c, d, e, f, g, h, i) are capable of hybridizing to one another.

The subsequences are joined together by linkers. The linkers are designed and selected such that the subsequences of each oligonucleotide form around each face of the polyhedron. Thus, in the case of a regular tetrahedron, the linker acts as a hinge region and allows formation of an angle of approximately 60° between the subsequences in the oligonucleotide. In the case of a regular cube, the linker acts as a hinge region and allows formation of an angle of approximately 90° between the subsequences in the oligonucleotide. Any of the linkers discussed above can be used in accordance with the specific embodiment.

The oligonucleotides are designed such that each oligonucleotide comprises subsequences and linkers such that the oligonucleotide runs around a face of the polyhedron, and the subsequences can hybridize to subsequences of the oligonucleotides along the shared edge of the adjacent faces. The oligonucleotide can comprise fewer or more subsequences together with linkers such that the polyhedron has hybridized, double stranded sequences along the edge of each face with linker sequences where required. For example, in a tetrahedron, the oligonucleotide can comprise four subsequences having linkers therebetween, each linker forming the vertices of the face, in which the first and fourth sequences of the oligonucleotide hybridize to sequences along one of the shared edges of the face. For example, the first oligonucleotide can be represented as having the sequence (a1, b, c, a2) or (b1, c, a, b2) or (c1, a, b, c2), the subsequences (a1, a2) being the same as the sequence (a) as previously described.

Any of the oligonucleotides and subsequences discussed above can be used in accordance with the specific embodiment. The subsequences are selected such that each subsequence hybridizes to a subsequence or subsequences of the oligonucleotide at the shared edge of the adjacent face.

In accordance with the present invention, the nucleic acid polyhedron can be formed in a simple procedure. Firstly, the oligonucleotides and the moiety are mixed together, preferably in equimolar quantities in the presence of a buffer. Any suitable buffer can be used, in particular those containing a counter ion such as Na+, Ca2+ or Mg2+. For example, TE (Tris-EDTA) with Magnesium ions can be used (referred to herein as TEM). Any suitable concentration of oligonucleotides and moiety can be used. In some embodiments, the nucleic acid polyhedron spontaneously forms once the oligonucleotides have been mixed. In such embodiments, there is no need for the heating and cooling steps to be carried out.

In other embodiments, the mixture in buffer solution is treated to remove all secondary structure of the oligonucleotides and in particular to denature any double stranded nucleic acid present in the mixture. Such treatment can involve heating the mixture, for example to a temperature above the T melt of the subsequences, such as 50° C. to 100° C. Preferably, the mixture is heated to 50 to 80° C., most preferably 50 to 60° C. Preferably, heat is applied over a period of 30 seconds to 10 minutes, preferably, 1 to 5 minutes, such as 2 minutes. Longer heating, or treatment times can also be used.

If the mixture is heated, it is then optionally treated in order to allow the subsequences to hybridize or anneal to each other. Preferably, the mixture is cooled, for example to 1 to 40° C., preferably to room temperature of about 20° C. to allow hybridization to occur. The treatment can be carried out over a period of about 30 seconds to 10 minutes, for example, 1 to 5 minutes, such as 2 minutes. Longer hybridization times can also be used such as 10 mins to 1 hour, up to 24 hours.

The temperatures for denaturation or removal of any secondary structure and for hybridization can be varied depending on the nature of the buffer solution being used, and the length of the oligonucleotides and subsequences thereof, taking into account the T melt of the selected sequences.

The mixture can be subjected to a rapid drop in temperature from the denaturing temperature to allow hybridization to occur. For example, cooling to the selected final temperature can occur within 5 minutes, preferably under 3 minutes, for example between 30 seconds and 2 minutes.

In some embodiments, the heating and cooling steps are carried out. If the heating and cooling steps are carried out, the method comprises:

(a) providing one or more oligonucleotides, each oligonucleotide comprising subsequences that hybridize to subsequences on the same oligonucleotide or a different oligonucleotide to allow the oligonucleotide(s) to form the polyhedron;

(b) mixing the oligonucleotide(s) of (a) and the moiety in a buffer solution; and

(c) subjecting the mixture of (b) to a heat treatment comprising a heating step to denature the oligonucleotide(s) and a cooling step to anneal the oligonucleotide(s) to allow them to hybridize to form thereby a nucleic acid polyhedron;

wherein steps (b) and (c) are carried out in such a manner that the moiety becomes contained within the nucleic acid polyhedron.

In other embodiments, the heating and cooling steps are not carried out. If the heating and cooling steps are not carried out, the method comprises:

(a) providing one or more oligonucleotides, each oligonucleotide comprising subsequences that hybridize to subsequences on the same oligonucleotide or a different oligonucleotide to allow the oligonucleotide(s) to form the polyhedron; and

(b) mixing the oligonucleotide(s) of (a) and the moiety in a buffer solution to allow the oligonucleotide(s) to hybridize to form thereby a nucleic acid polyhedron;

wherein step (b) is carried out in such a manner that the moiety becomes contained within the nucleic acid polyhedron.

The mixing step and the heating and cooling steps of they are performed are carried out in such a manner that the moiety is contained inside the polyhedron. The moiety is preferably attached to or bound to one of the oligonucleotides before it is mixed with the other oligonucleotides forming the nucleic acid polyhedron. The moiety can be attached to or bound to the oligonucleotide in any manner. For example, the moiety can be attached to or bound to the oligonucleotide through an ionic interaction or a covalent linkage. The moiety may be attached or bound to the oligonucleotide via one or more linker molecules. Suitable attachment or binding methods include covalently linking the moiety to the oligonucleotide using bifunctional cross-linker molecules, an aptamer or a poly-histidine tag. The attachment or binding may be reversible or irreversible. The link attaching the moiety to the oligonucleotide can be broken or cleaved after the polyhedron has formed.

The moiety can be attached to or bound to a nucleotide at either end of the oligonucleotide or to a nucleotide in the middle of the oligonucleotide. By attaching or binding the moiety to a selected nucleotide, the moiety may be specifically associated with the inside of the nucleic acid polyhedron. If the oligonucleotide edges of the polyhedron are nucleic acid double helices that cannot substantially rotate around their axes, shifting the conjugation position of the moiety by one nucleotide not only shifts the moiety along the edge but also phases it around the edge of the polyhedron. Every step along the oligonucleotide shifts the moiety conjugation position by 0.3 nm and 35°. The moiety can therefore be associated with the inside of the nucleic acid polyhedron by attaching it before hybridization to a nucleotide that is positioned on the inside of the nucleic acid polyhedron after the oligonucleotides have hybridized or annealed to each other. The final position of a nucleotide in a nucleic acid polyhedron can be determined. The position of each nucleotide is typically fixed because the vertices of the polyhedron prevent rotation of the edges. The moiety can be associated with the inside of the nucleic acid polyhedron by attaching or binding it to a nucleotide that ends up on the inside of the nucleic acid polyhedron.

Alternatively, any other method of containing the moiety inside the nucleic acid polyhedron can be used in accordance with the present invention. For example, the mixing step and the heating and cooling steps if they are performed can be carried out in the presence of compounds containing hydrophobic groups, such as lipids, which cause the moiety to be contained inside the polyhedron. Compounds containing hydrophobic groups can generate a hydrophobic environment in the interior of the polyhedron during its formation. This environment can cause a hydrophobic moiety to become contained inside the nucleic acid polyhedron. Suitable lipids are well-known in the art.

The nucleic acid polyhedron so formed may be modified in order to allow the nucleic acid polyhedra to be joined together or connected to other objects. The oligonucleotides forming the nucleic acid polyhedron may be provided with overhangs, extension sequences or other chemical moieties which may be used in such attachments.

Such chemical moieties may be conjugated to or bound to the oligonucleotides through suitable linkages, such as covalent bonds to allow the attachment or conjugation of the polyhedra to other polyhedra or other nanostructures. Similarly, one or more of the oligonucleotides may be extended to incorporate an extension sequence which can be used to bind the nucleic acid polyhedron to other polyhedra or nanostructures.

The polyhedron once formed may also be subjected to a ligation reaction in order to essentially join each nicked edge of the polyhedron through ligation of the free oligonucleotide ends.

### Cross-Bridges

In some embodiments, the polyhedron of the invention can comprise at least one nucleic acid cross-bridge. A nucleic acid cross-bridge is a length of nucleic acid that crosses one or more of the faces of the polyhedron thereby splitting the face into two or more sections and reducing the area of the face. A nucleic acid cross-bridge is also a length of nucleic acid that crosses the inside of the polyhedron between two edges thereby splitting the inside of the polyhedron into two or more sections. The nucleic acid polyhedron can comprise at least 1, at least 2, at least 3, at least 4 or at least 5 nucleic acid cross-bridges across at least 1, at least 2, at least 3, at least 5, at least 10, at least 12 or all of its faces. The nucleic acid polyhedron can contain one or more nucleic acid cross-bridges that cross the face of the polyhedron and/or one or more cross-bridges that cross the inside of the polyhedron.

In one embodiment, the cross-bridge is formed by covalently linking (by ligation or in synthesis) two of the oligonucleotides forming the nucleic acid polyhedron before the polyhedron is formed and forming the polyhedron from the ligation product and the other oligonucleotide strands. In another embodiment, the polyhedron of the invention is formed as discussed above and is selected to include overhangs. A cross-bridge strand(s) (splint strand), which is designed to hold the overhangs in place, can be added before or after the heat treatment. The cross-bridge strand hybridizes to the overhangs and the overhangs can be ligated to each other, for example by DNA Ligase. A variation of this approach would be to use overhangs that hybridize to each other directly to form a cross-bridge without the need for a separate cross-bridge strand.

### Triggered Release

The nucleic acid polyhedron of the present invention may be used as a delivery vehicle for the moiety. The structure of the nucleic acid polyhedron is preferably modified to allow for release of or for control of access to the moiety in response to a trigger or synthesis, for example in the presence of an enzyme or in the presence of another nucleic acid molecule. The nucleic acid polyhedron is more preferably modified to allow for release of the moiety in the presence of a specific DNA sequence, RNA sequence, protein or small molecule. If the moiety is attached or bound to the polyhedron, the structure of the nucleic acid polyhedron is preferably modified to allow for access to the moiety, for example the polyhedron may open or break in the presence of an enzyme or in the presence of another DNA molecule thereby granting access to the moiety contained therein.

One or more of the edges of the polyhedron can be designed to change length or break in response to a target nucleic acid, such as an oligonucleotide or mRNA, or another target molecule, such as a protein or small molecule, binding to an aptamer domain. The aptamer can be DNA or RNA. In one embodiment, the aptamer is part of one or more of the edges of the polyhedron and its ends are further apart when bound to the target molecule than in the absence of the target molecule. In another embodiment, one or more single-stranded sections of one or more of the edges are designed to hybridize with a domain of the aptamer in such a way that aptamer binding opens or breaks the edge.

In a preferred embodiment, one or more of the edges and/or vertices of the nucleic acid polyhedron can be designed to contain a short hairpin loop. For example, at least 1, at least 2, at least 3, at least 4 or all of the edges and/or vertices may contain a hairpin loop. An edge or a vertex can contain 1, 2, 3, 4 or 5 or more hairpin loops. The loop preferably contains at least 3, at least 4, at least 5 or at least 10 nucleotides, such as from 5 to 30 or from 10 to 20 nucleotides, with a stem of at least 3, at least 4, at least 5 or at least 10 base pairs, such as from 5 to 30 or from 10 to 20 base pairs. The loop can be designed such that it hybridizes to a specific “trigger” or “fuel” DNA sequence. For example, the loop may be completely complementary to the “trigger” or “fuel” sequence or may have one, two or more mismatches. Exposure of the polyhedron to the “trigger” or “fuel” sequence results in the loop being opened out as it hybridizes to the “trigger” or “fuel” sequence. The resulting increase in length of one or more of the polyhedron edges results in the expansion of one or more of the faces of the polyhedron. The extent of expansion can be designed to allow for release of a moiety contained in the polyhedron. The opening of the loop and expansion of the face may be reversed by exposing the polyhedron to a DNA sequence that hybridizes to the “trigger” or “fuel” sequence i.e. an “anti-trigger” or “anti-fuel” sequence. For example, the “anti-trigger” or “anti-fuel” sequence may be completely complementary to the “trigger” or “fuel” sequence or may have one, two or more mismatches.

### Incomplete Nucleic Acid Polyhedron

The nucleic acid polyhedron can be incomplete. The nucleic acid polyhedron may lack one or more edges and/or vertices. For example, a nucleic acid tetrahedron or a nucleic acid cube may lack one edge and/or vertex and a nucleic acid octahedron may lack one or two edges and/or vertices.

### Linked Nucleic Acid Polyhedra Having a Moiety Contained Therein

The nucleic acid polyhedron containing a moiety can be linked to one or more moiety-containing and/or non-moiety-containing nucleic acid polyhedra. Hence, the present invention also provides a structure comprising two or more nucleic acid polyhedra, wherein at least one of the polyhedra has a moiety contained therein. The nucleic acid polyhedra may be the same or different. The structure preferably comprises at least 2, at least 3, at least 4, at least 5, at least 10, at least 20 or at least 50 nucleic acid polyhedra that are the same or different. Examples include structures comprising two regular tetrahedra, a regular tetrahedron and an irregular tetrahedron, a dipyramid, three regular octahedra and five irregular cubes. The nucleic acid polyhedra may be linked together in arrays, chains or other shapes. The structure is preferably a dipyramid which comprises two nucleic acid tetrahedra wherein each is joined through one of its faces.

One, some or all of the nucleic acid polyhedra in the structure can be selected to contain a moiety. Preferably, at least 1, at least 2, at least 3, at least 4, at least 5, at least 5, at least 10, at least 20 or at least 50 of the nucleic acid polyhedra contains a moiety. The nucleic acid polyhedra may contain the same moiety or different moieties.

The nucleic acid polyhedra may be linked through their vertices, through their edges or through their faces. The nucleic acid polyhedra may be linked by any suitable linkage, such as covalent bonds, preferably via extension sequences or other chemical moieties present in the nucleic acid polyhedra. In some cases, the nucleic acid polyhedra are linked by one or more linker molecules, such as DNA.

Nucleic Acid Polyhedron with a Moiety Attached on the Outside

As will be appreciated, the method of the present invention may also be used to make a nucleic acid polyhedron having a moiety attached to the outside of the polyhedron. The moiety can be associated with the outside of the nucleic acid polyhedron by attaching it to or binding it to a nucleotide that is positioned on the outside of the nucleic acid polyhedron after the oligonucleotides have hybridized or annealed to each other. The final position of a nucleotide in a nucleic acid polyhedron can be determined.

The present invention therefore also provides a nucleic acid polyhedron having a moiety attached to the outside of the polyhedron and a method of making it. The nucleic acid polyhedron can be any of the nucleic acid polyhedra discussed above. The moiety can be any of moieties discussed above.

The moiety is attached to or bound to an external edge or an external vertex of the nucleic acid polyhedron. The moiety is preferably attached to or bound to the outside of the nucleic acid polyhedron by an ionic interaction or a covalent linkage. For example, the moiety may be attached to or bound to the outside of the cage using one or more linker molecules, such as bifunctional cross-linker molecules, aptamers or poly-histidine tags.

The nucleic acid polyhedron having a moiety attached to the outside of the polyhedron can also be linked to one or more nucleic acid polyhedra as discussed above.

### Uses of the Nucleic Acid Polyhedron

A nucleic acid polyhedron having a metal-containing or a mineral-containing moeity contained therein or attached thereto may be used as a template for or a component of nanoelectronic and nanoelectromechanical systems.

The nucleic acid polyhedron of the present invention may also be used as a delivery vehicle for the moiety. For example, the nucleic acid polyhedron may be used for delivery of pharmaceutical substances such as proteins, DNA, RNA, miRNA, siRNA and drugs. The structure of the polyhedrons can be modified to allow for release of the moiety for delivery as discussed above

Nucleic acid polyhedrons having protein or a nucleic acid contained therein or attached thereto may also be used in vaccine formulations. The nucleic acid polyhedrons can be used to deliver nucleic acid and/or a protein epitope. The nucleic acid polyhedrons may be designed to carry nucleic acid encoding a desired epitope. The following Examples illustrate the invention.

### EXAMPLES

### Example 1

### Making a DNA Tetrahedron

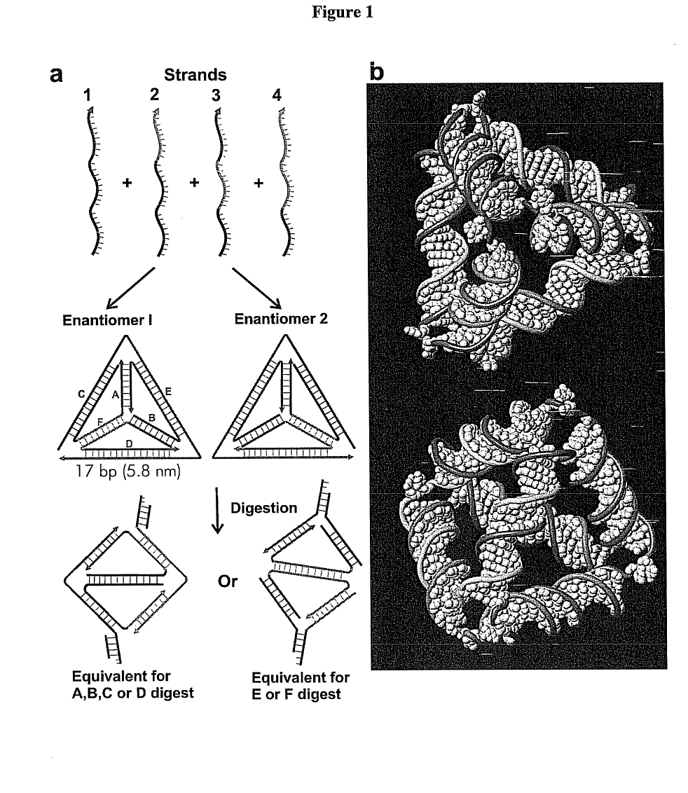

FIG. 1a illustrates our synthetic scheme. Each tetrahedron is assembled from four 55-base oligonucleotides (Table 1, strands 1-4).

TABLE 1
Oligonucleotides and Edge-Cutting Restriction Enzymes

Oligonucleotides*:
Strand 1:

(SEQ ID NO: 1)

ACATTCCTAAGTCTGAAACATTACAGCTTGCTACACGAGAAGAGCCGCCA
TAGTA
Strand 2:

(SEQ ID NO: 2)

TATCACCAGGCAGTTGACAGTGTAGCAAGCTGTAATAGATGCGAGGGTCC
AATAC
Strand 3:

(SEQ ID NO: 3)

TCAACTGCCTGGTGATAAAACGACACTACGTGGGAATCTACTATGGCGGC
TCTTC
Strand 4:

(SEQ ID NO: 4)

TTCAGACTTAGGAATGTGCTTCCCACGTAGTGTCGTTTGTATTGGACCCT
CGCAT
Strand 5:

(SEQ ID NO: 5)

ACATTCCTAAGTCTGAAACTTCCCACGTAGTGTCGTTTGTATTGGACCCT
CGCAT
Strand 6:

(SEQ ID NO: 6)

TTCAGACTTAGGAATGTGCATTACAGCTTGCTACACGAGAAGAGCCGCCA
TAGTA
Enzymes:
Edge A: Dde I. Edge B: Aci I. Edge C: Scr Fi.
Edge D: Sau96 I. Edge E: Alu I. Edge F: HypCh4 IV.

Each of the six edges of the tetrahedron is made from one of six 17-base ‘edge subsequences’ hybridized to its complement. Edge subsequences were designed to minimize the strength of undesirable interactions between them. Each strand contains three of these subsequences, or their complements, separated by two-nucleotide ‘hinges’ which are designed to remain unhybridized. Hinges were incorporated in the design to ensure that the vertices of the construct have sufficient flexibility to accommodate an angle of 60° between adjacent edges. The four component oligonucleotides are thus designed to self-assemble to form a regular tetrahedron (FIG. 1b) consisting of double-stranded edges connected to each other through two-nucleotide hinges. Each oligonucleotide runs round one of the four faces and is hybridized to the three oligonucleotides running round the adjacent faces at the shared edges. Each vertex is a nicked three-arm junction. As the edges are distinguishable (each has a different sequence) each tetrahedron is chiral; the two possible diastereomers are illustrated in FIG. 1a.

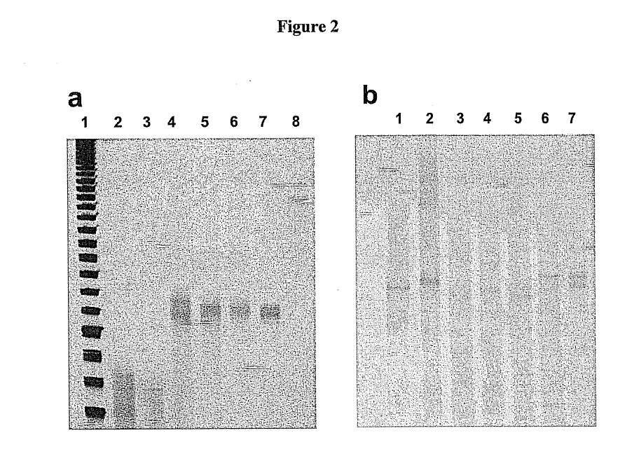

To form the tetrahedra equimolar quantities (1 μM) of oligonucleotides 1 to 4 are combined in TEM buffer (10 mM Tris, 1 mM EDTA, 20 mM MgCl₂, pH 8.0). The temperature is raised to 95° C. for two minutes, and then reduced to 20° C. over 2 minutes in a Techne thermocycler. A single-band product is visible on 12.5% PAGE gel (FIG. 2a, lane 8). By excluding other possibilities by means of control experiments described below we infer that this single product consists of DNA tetrahedra as designed.

Control experiments explore the effects of omitting components or of cutting edges, and the stoichiometry of the complex. FIG. 2a demonstrates that the presence of all four oligonucleotides is necessary for the formation of the product; the absence of additional bands in lane 8 demonstrates that all four are incorporated stoichiometrically. To investigate the possibility that the product band contains not one but two or more copies of each oligonucleotide we have assembled tetrahedra in the presence of oligonucleotides with 12 base pair 3′ extensions as gel-shift labels (the extensions are designed to remain unhybridized). No products containing mixtures of original and extended oligonucleotides were detected, confirming that the construct contains a single copy of each oligonucleotide, as designed. FIG. 2b demonstrates that each of the six double-stranded edges is present in the product. Each edge is designed to contain a unique double-stranded restriction site that can be recognised and cut specifically by a restriction endonuclease: Each digestion results in a shift in the mobility of the band, but no fragmentation, which is consistent with the design of the tetrahedron (FIG. 1a). It is interesting to note that while the digestion of edges A-E are essentially complete, a small amount of the original band remains when edge F is cut. We cannot rule out steric hindrances as a possible source of enzymatic inefficiency, and we know that only one of the diastereomers is formed.

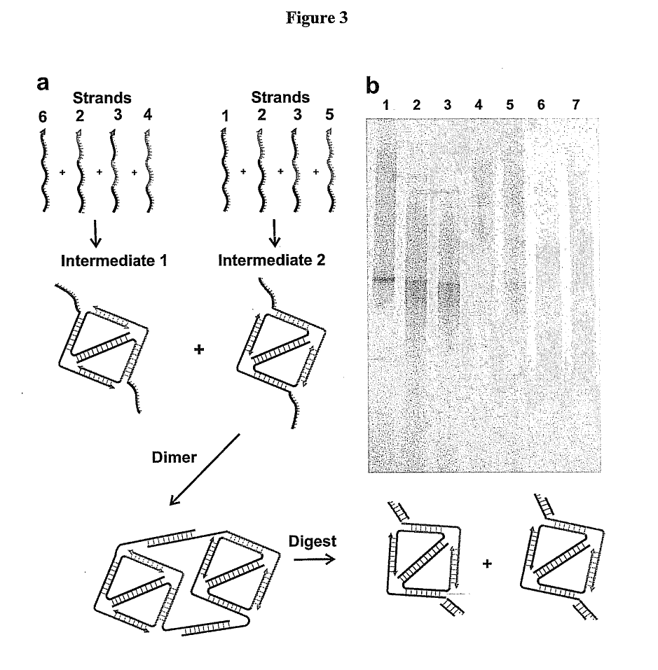

To further investigate the possibility that the product band corresponds to a larger complex we replaced one complementary pair of edge subsequences contained in strands 1 and 4 to produce two intermediate constructs that cannot close to form tetrahedra but that together can form dimers or higher multimers. FIG. 3a illustrates this scheme. FIG. 3b shows that each intermediate produces a smeared band that has higher mobility than the tetrahedral band. When combined the two intermediates generate complexes with a wide range of mobilities, but no band corresponding to the normal tetrahedron. However, when the dimer is digested with the restriction endonuclease specific to the linking arms (both of which contain the same recognition site), the digestion product is identical to that of the corresponding edge digest of the tetrahedron. This confirms that multimers are not produced in significant amounts in the original tetrahedron synthesis.

We have investigated the necessity of incorporating the two-base-pair hinges at each vertex. A single-base-pair hinge gives similar results, but no tetrahedral band is seen when no linker is present (data not shown).

The DNA tetrahedron is the simplest possible DNA polyhedron, and is particularly suitable as a building block for extended DNA nanostructures as its braced geometry, consisting entirely of triangles, limits the range of configurations that it can adopt.

### Example 2

### Making a DNA Octahedron

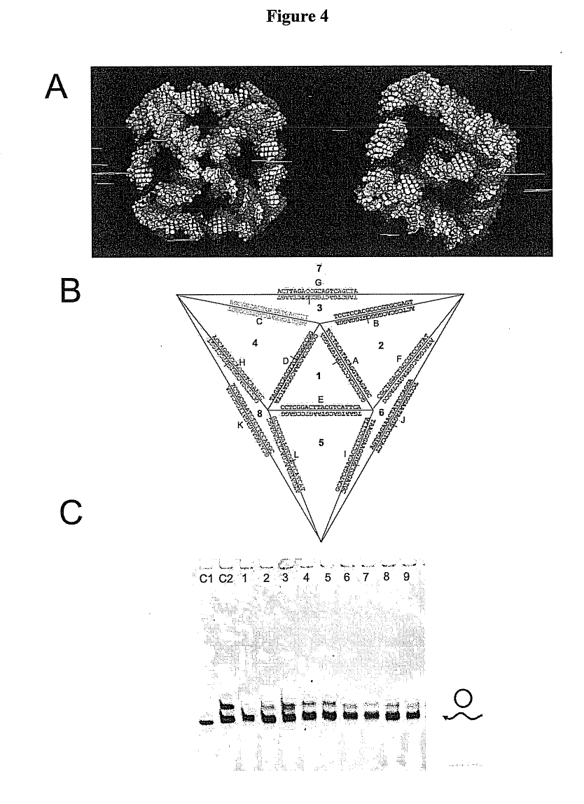

A DNA octahedron was made from eight oligonucleotides using a method analogous to that described in Goodman et al. Science 2005 310, 1661-1665. The octahedron is shown in FIG. 4.

### Example 3

### Making a DNA Bipyramid

In this Example, we demonstrate the formation of a trigonal bipyramid via one-step assembly. All oligonucleotides were supplied (with and without 5′ phosphorylation) by Integrated DNA Technologies. Sequences were designed using NANEV^{S1}. The following sequences were used.

a:

(SEQ ID NO: 7)

AGGCAGTTGAGACGAACATTCCTAAGTCTGAAATTTATCACCCGCCATAG
TAGACGTATCACC
b:

(SEQ ID NO: 8)

CTTGCTACACGATTCAGACTTAGGAATGTTCGACATGCGAGGGTCCAATA
CCGACGATTACAG
c:

(SEQ ID NO: 9)

GGTGATAAAACGTGTAGCAAGCTGTAATCGACGGGAAGAGCATGCCCAT
CCACTACTATGGCG
A:

(SEQ ID NO: 10)

CTGCGCGGATGACTCAACTGCCTGGTGATACGATCTAGTCTCTACGTCAA
GTAAGAACCTTAG
B:

(SEQ ID NO: 11)

CCTCGCATGACATCCGCGCAGCTAAGGTTCAAAGTTCCTGCCGCTTCACG
GACGGTATTGGAC
C:

(SEQ ID NO: 12)

CTCTTCCCGACCGTGAAGCGGCAGGAACTTATACTTGACGTAGAGACTAG
AAGGATGGGCATG

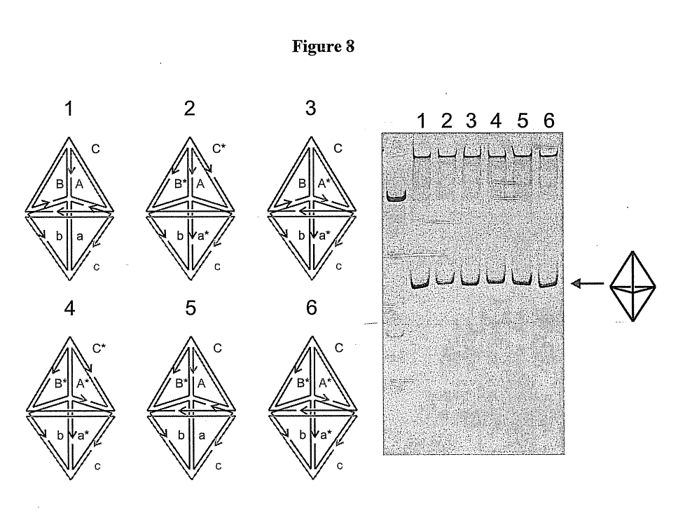

Additional sequences (supplied with 5′ phosphorylation) used for the bipyramids shown in FIG. 8:

a*:

(SEQ ID NO: 13)

CTAAGTCTGAAATTTATCACCCGCCATAGTAGACGTATCACCAGGCAGTT
GAGACGAACATTC
A*:

(SEQ ID NO: 14)

CTGGTGATACGATCTAGTCTCTACGTCAAGTAAGAACCTTAGCTGCGCGG
ATGACTCAACTGC
B*:

(SEQ ID NO: 15)

GCTTCACGGACGGTATTGGACCCTCGCATGACATCCGCGCAGCTAAGGTT
CAAAGTTCCTGCC
C*:

(SEQ ID NO: 16)

GAGACTAGAAGGATGGGCATGCTCTTCCCGACCGTGAAGCGGCAGGAAC
TTATACTTGACGTA

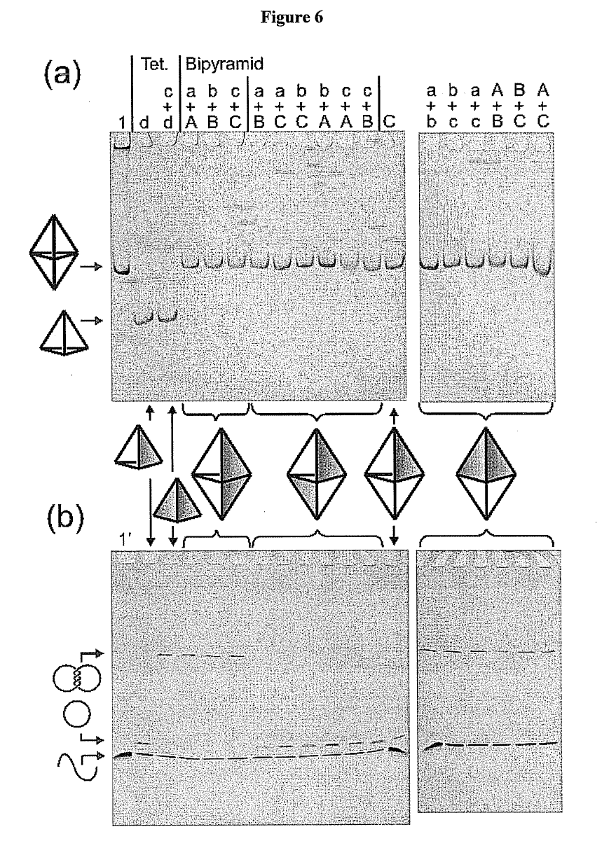

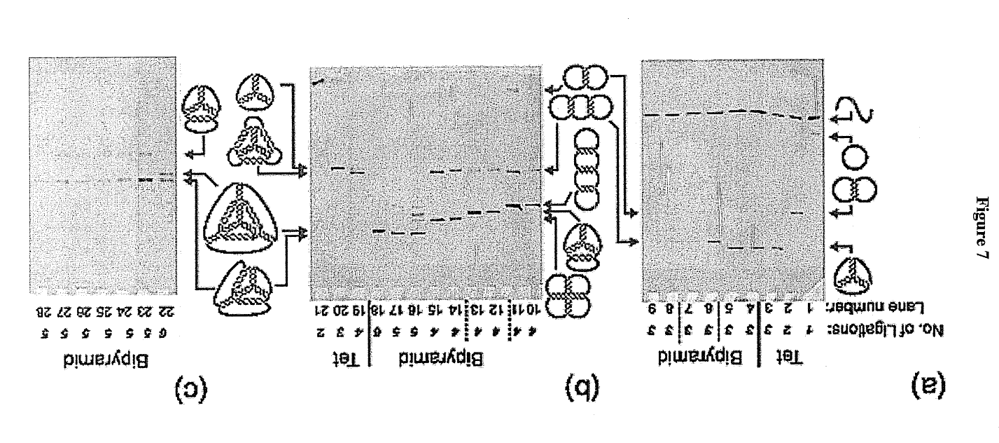

Tetrahedron controls in FIGS. 6 and 7 were formed using oligonucleotides a, b, c (see above) and d:

d:

(SEQ ID NO: 17)

CCTCGCATGACTCAACTGCCTGGTGATACGAGGATGGGCATGCTCTTCCC
GACGGTATTGGAC

To determine the yield of assembly, we have analyzed the unpurified product of the self-assembly step. The product was run on a 6% native gel (acrylamide:bisacrylamide ratio: 19:1) and stained with SYBR Gold (Molecular Probes/Invitrogen). Using a Pharos FX Plus Molecular Imager (Bio-Rad) and Quantity One analysis software (Bio-Rad), we have determined the ratio between the intensity of the bipyramid band and the total intensity in the lane (minus the background). We determined a bipyramid yield of approximately 40%. Yield is sensitive to accurate stoichiometry: the difficulty of adjusting the stoichiometry of the six strands of the bipyramid may contribute to the difference between the yields obtained in bipyramid and tetrahedron syntheses.

All native polyacrylamide gel electrophoresis (PAGE) gels shown contain 6% acrylamide (29:1 acrylamide:bisacrylamide ratio) and were run in TAE buffer. DNA was visualized by silver staining.

Denaturing gels contain 7M Urea and 0.1% SDS and have stacking and separating layers with acrylamide content and acrylamide:bisacrylamide ratios as follows: FIG. 6 and FIG. 7(a) 5%, 12% (29:1); FIG. 7(b): 5%, 8% (19:1); FIG. 7(c): 5%, 6% (19:1). Samples were combined 1:1 with a loading buffer containing 7 M urea and 3% SDS and held at 95 degrees for 5 minutes before loading.

Oligonucleotides to be ligated were purchased with 5′ phosphorylation (Integrated DNA Technologies). After annealing, bipyramid samples were ligated by incubation with T4 DNA ligase (New England Biolabs) at room temperature. Ligated samples were gel purified on native 6% PAGE gels in TAE buffer. The bipyramid band was cut out of the gel and recovered using the crush and soak method. Gel slices were crushed, covered with buffer, soaked overnight and spun on Millipore Ultrafree-MC HV centrifugal filters (0.45 μm) to remove the crushed gel.

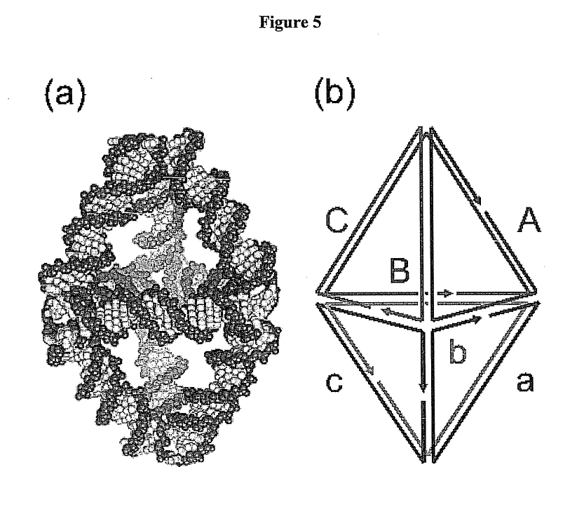

The structure of the DNA bipyramid is illustrated in FIG. 5. Six strands of DNA (synthetic oligonucleotides) each run around one of the faces and hybridize to neighbouring strands to form nine 20-basepair edges (two turns of the double helix, 7 nm). Single, unpaired adenosine nucleotides connect neighbouring edges at the vertices. Six of the nine edges contain nicks (breaks in the DNA backbone) where the 5′ and 3′ ends of one strand meet. FIG. 5 shows the positions of the nicks in the bipyramid whose formation is reported below; other nick positions are also compatible with efficient assembly (FIG. 8).

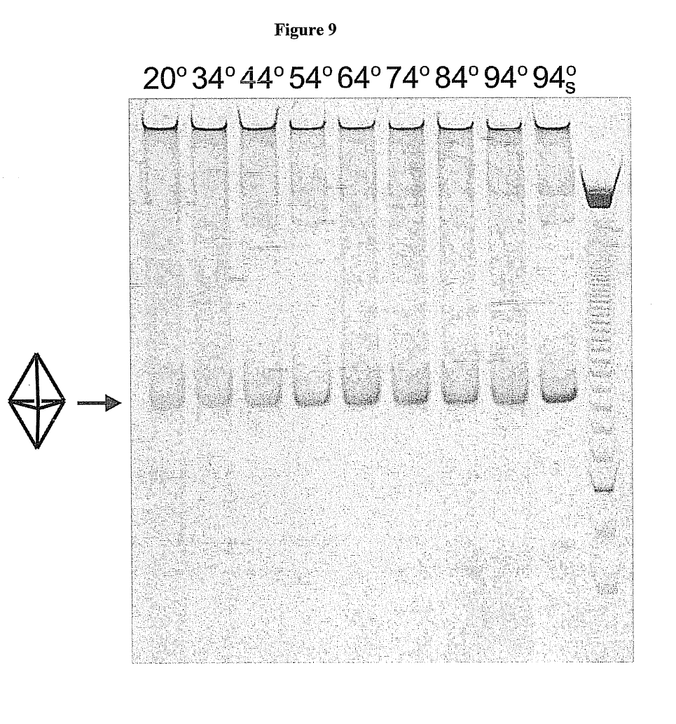

The bipyramid is formed in a single annealing step. The six strands are combined in equimolar amounts (⅙^{th} uM each in the final solution) in 10 mM Tris buffer (pH 8) with 5 mM MgCl₂ and heated to 95° C. then cooled to room temperature. Cooling over ˜15 minutes leads to slightly higher yield than the 30 s fast cooling protocol used for DNA tetrahedra (R. P. Goodman, I. A. T. Schaap, C. F. Tardin, C. M. Erben, R. M. Berry, C. F. Schmidt, A. J. Turberfield, Science 2005, 310, 1661). High yield is obtained for all starting temperatures above 50° C., and there is significant yield even when the reaction mixture is simply incubated at room temperature (FIG. 9). FIG. 6 shows polyacrylamide gel electrophoresis (PAGE) analysis of the products of the single-step annealing process. The first lane in FIG. 6(a) contains all products: a single high-intensity band corresponds to the bipyramid; there are by-products of higher molecular weight. The identity of the bipyramid was confirmed by investigating the topological relationships between component strands, as described below.

Fifteen different bipyramids were formed, corresponding to all combinations of two 5′-phosphorylated strands and four unphosphorylated strands. After cooling, samples were incubated with T4 DNA ligase (New England Biolabs). This enzyme will ligate (covalently join) a phosphorylated 5′ end to a 3′end if the two free ends are held together by hybridization to a common ‘splint’ strand: if the bipyramid forms as designed, the 5′ and 3′ ends of each phosphorylated strand will be ligated to form a closed circle. If and only if the two phosphorylated strands run along a common edge of the bipyramid then the two circles will be linked (with linking number 2). The bipyramids were gel-purified and analyzed by both native and denaturing PAGE. FIG. 6a shows that all 15 purified DNA bipyramids migrate with approximately the same mobility in a native gel. (Two control lanes contain purified 20-basepair DNA tetrahedra (R. P. Goodman, I. A. T. Schaap, C. F. Tardin, C. M. Erben, R. M. Berry, C. F. Schmidt, A. J. Turberfield, Science 2005, 310, 1661) with one and two phosphorylated strands ligated.) FIG. 6b shows the same samples on denaturing gels in which the base pairing between DNA strands is disrupted. Bands corresponding to unligated strands, single closed circles and linked pairs of circles can be identified by comparison with control lane 1′ and the partially-ligated tetrahedron controls respectively. As expected, ligation of any of the nine pairs of strands that are designed to share an edge leads to the formation of linked circles and ligation of the six pairs of strands which are not designed to share an edge forms unlinked circles. This result confirms that the topological relationship between each pair of strands is as designed.

Ligation of more than two strands creates more complex catenanes. For example: ligation of strands a, b and c creates the same pattern of three interlinked circles as is formed by ligation of any three strands in a DNA tetrahedron (R. P. Goodman, I. A. T. Schaap, C. F. Tardin, C. M. Erben, R. M. Berry, C. F. Schmidt, A. J. Turberfield, Science 2005, 310, 1661); ligation of strands a, c and A creates a linear chain; ligation of strands a, B and C creates two linked circles and one unlinked circle. Within the bipyramid there are examples of three different ways to link (or not link) three circles, three ways to link four circles and one way to link five circles. We have created each of these linkage patterns twice, by ligating different subsets of strands that are expected to produce the same pattern. The pattern of six linked circles can be made in only one way, by ligating all six strands. FIG. 7(a,b) shows denaturing PAGE analysis of these catenanes, which were purified from a native gel. (Faster bands correspond to a smaller numbers of linked circles created by ligation failures.) Catenanes containing the same number of circles differently linked have different mobilities, but the products of ligation of different subsets of strands designed to have the same pattern of linkage have the same mobility, as expected.

FIG. 7(c) is a low-percentage denaturing polyacrylamide gel that can resolve the small mobility difference between the bipyramid in which all six nicks have been ligated and the six possible constructs with any one of the six strands not ligated. Six interlinked circles have a higher mobility than five.

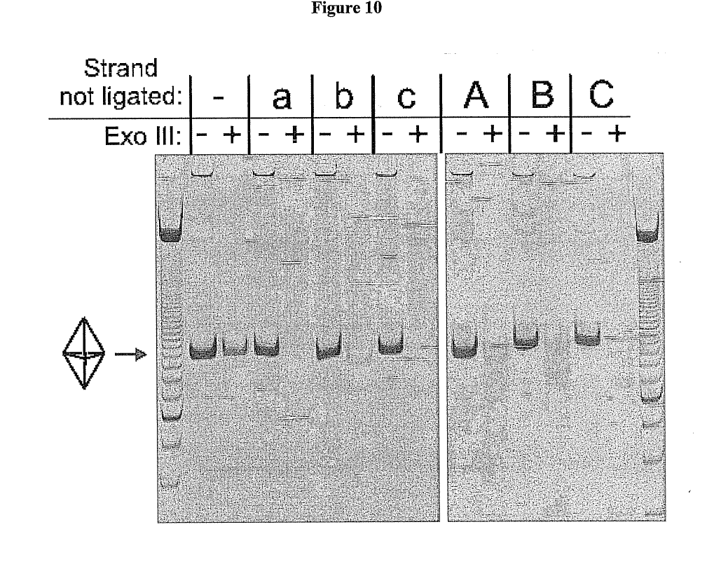

The structure of the bipyramid has also been confirmed by incubation with exonuclease III (New England Biolabs) which digests linear oligonucleotides. The bipyramid band survives enzymatic digestion, but only if all six nicks have been ligated, confirming that this construct contains all six strands and that all are circular, as designed (FIG. 10).

In summary, we have demonstrated that a trigonal bipyramid with 20-basepair edges can be formed in high yield in a single self-assembly step from six DNA strands. We have used combinations of ligation and denaturing PAGE to demonstrate that the topological relationships between strands are as designed. This is only the fifth self-assembled DNA polyhedron whose formation has been demonstrated: its synthesis shows that the high-yield, single-step assembly developed for DNA tetrahedra can be extended to larger polyhedra.

### Example 4

### Encapsulating a Protein Inside a DNA Tetrahedron

The DNA tetrahedra used here were made by self-assembly of four oligonucleotides s1-s4 as described in Goodman et al Science 2005 310, 1661-1665. Each edge of the tetrahedron is a 20-basepair double helix. The two oligonucleotides forming an edge separate to link it, though unpaired single-nucleotide spacers, to the two edges that it meets at a vertex: these connections prevent rotation of the edge about its axis. In the diastereomer formed by the stereoselective assembly process the edges are oriented with the major grooves inwards at the vertices (Goodman et al. Science 2005 310, 1661-1665). Each oligonucleotide runs around one face: four of the six edges of the tetrahedron contain nicks where the ends of an oligonucleotide meet. These nicks can be healed by ligation or used as sites for chemical modification.

Horse-heart holo-cytochrome c was conjugated to the 5′ end of oligonucleotide s1 through a surface amine before tetrahedron assembly. The 63-mer s1 with a 5′ C₆-amino modification was supplied by Integrated DNA Technologies. s1 had the sequence: TCACCAGGCAGTTGAGACGAACATTCCTAAGTCT GAAATTTATCACCCGCCATAGTAGACGTA (SEQ ID NO: 18). The 5′ end of s1 carries a 5′ C₆-amino modification for conjugation to the protein. The sequence given here leads to a protein attachment at base number 5 along the edge formed s1 and s4. The other versions of s1 are obtained by transferring nucleotides from the 5′ to the 3′ end.

Holo-cytochrome c from equine heart was supplied by Sigma. The two molecules were conjugated by means of two heterobifunctional cross-linkers, sulfosuccinimidyl 4-(N-maleimidomethyl)cyclohexane-1-carboxylate (sulfo-SMCC) and N-succinimidyl 3-(2-pyridyldithio) propionate (SPDP) supplied by Pierce Biotechnology. Amino-modified oligos s1 were ethanol precipitated and re-suspended in phosphate buffer (100 mM Na₂HPO₄, 100 mM NaCl, pH 7.3) to 1 mM. The DNA solution was combined 1:2 with a saturated sulfo-SMCC solution (2.9 mg/ml) in phosphate buffer and incubated for 1 hour. Cytochrome c was dissolved in phosphate buffer to 1 mM, combined 25:2 with SPDP solution (5 mg/ml in DMSO) and incubated for 1 hour. Bio-Rad Micro Bio-Spin P-6 columns were used to remove excess cross-linkers from both solutions. Tris(2-carboxyethyl)phosphine hydrochloride (Sigma) was dissolved in phosphate buffer (10 mg/ml) and added 1:10 by volume to the cytochrome c solution to reduce the S—S bond in SPDP. After 30 minutes the DNA and cytochrome c solutions were combined (˜5:4 by volume) and incubated overnight at 4° C. N-Ethylmaleimide (Sigma) was dissolved in phosphate buffer (10 mg/ml) and added 1:25 by volume to the DNA-protein conjugate solution to bind any unreacted thiol groups. After 30 minutes a Bio-Rad Micro Bio-Spin P-6 column was used to transfer the conjugates into TM buffer (see below).

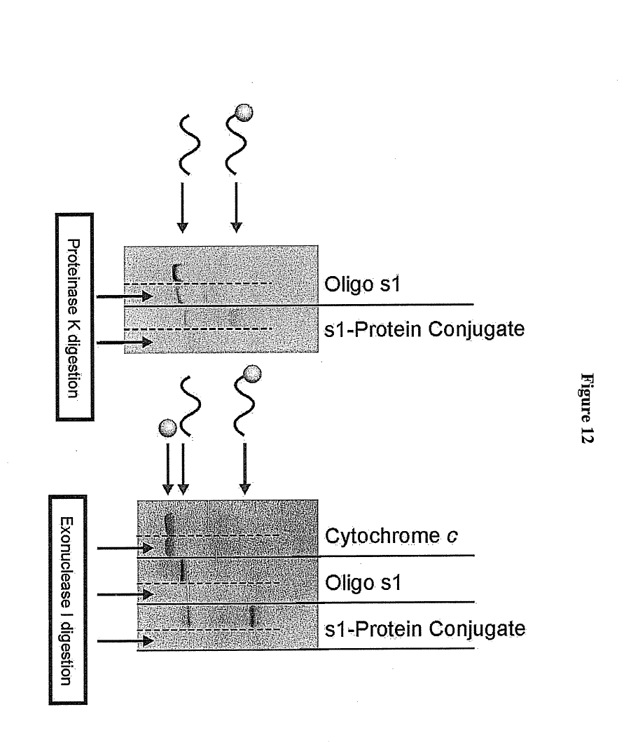

Confirmation of the identity of the s1-protein conjugate is shown in FIG. 12. Conjugates were gel-purified to remove proteins bound to more than one oligonucleotide by running them on native PAGE gels with 5% stacking buffer and 12% separating layers (29:1 acrylamide:bisacrylamide ratio) in Tris-glycine buffer. The desired bands were cut out of the gel and recovered using the crush and soak method

This protein-DNA conjugate was then combined with oligonucleotides s2, s3 and s4 to form tetrahedra with the protein attached to one edge.

s2 had the sequence:

(SEQ ID NO: 19)

CTTGCTACACGATTCAGACTTAGGAATGTTCGACATGCGAGGGTCCAATA
CCGACGATTACAG.
s3 had the sequence:

(SEQ ID NO: 20)

GGTGATAAAACGTGTAGCAAGCTGTAATCGACGGGAAGAGCATGCCCATC
CACTACTATGGCG.
s4 had the sequence:

(SEQ ID NO: 21)

CCTCGCATGACTCAACTGCCTGGTGATACGAGGATGGGCATGCTCTTCCC
GACGGTATTGGAC.

Equimolar amounts of the s1-cytochrome c conjugate and the 63-mers s2-s4 (supplied by Integrated DNA Technologies) were combined to a final concentration of 250 nm each oligonucleotide in Tris Buffer with 5 mM of divalent cations (either TM buffer: 10 mM Tris-HCl (pH 8), 5 mM MgCl₂ or TC buffer: 20 mM Tris-HCl (pH8), 5 mM (CaCl₂)). Annealing was performed by holding the mixture at 54° C. for 3 minutes followed by cooling to 4° C. If purification was desired, the tetrahedra were run on 8% native polyacrylamide gels (29:1 acrylamide:bisacrylamide ratio) in TAE buffer and the desired bands were cut out of the gel and recovered using the crush and soak method.

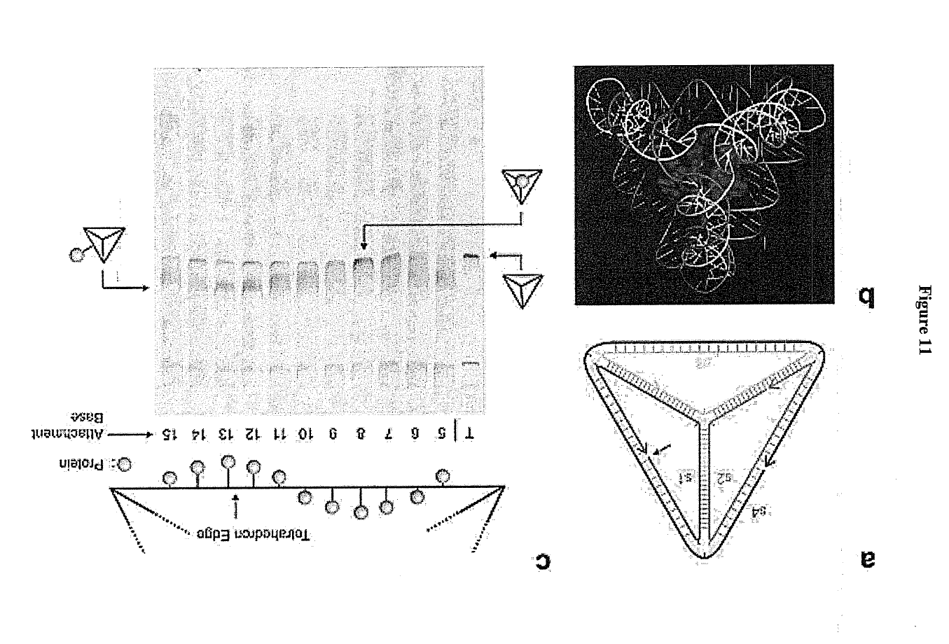

We can control the position of the protein relative to the cage by altering the sequence of s1: if one nucleotide is transferred from the 3′ end of s1 to the 5′ end then the attachment point of the protein, the nick in the edge formed by s1 and s4, moves 0.34 nm along the edge of the tetrahedron and rotates ˜35° clockwise about its axis. The position of the nick where the ends of oligonucleotide s1 meet therefore determines whether the protein is held on the inside or on the outside of the tetrahedron. FIG. 11b demonstrates how the protein can fit inside the central cavity of the DNA tetrahedron. The stereoselectivity of the tetrahedron assembly step is crucial to the success of this strategy for controlling the protein's position: an attachment point that is on the inside of one diastereomer would be on the outside of the other.

The native PAGE gel in FIG. 11c compares a group of 11 tetrahedra with cytochrome c conjugated at 11 consecutive positions along an edge, from the 5^{th} to the 15^{th} base (counting the unpaired base at the vertex as zero). The control lane labelled ‘T’ contains a tight band corresponding to a DNA tetrahedron without protein: this band is present in all other lanes (due to the presence of residual unconjugated oligonucleotides s1) and is a useful reference marker. A broader, slower band corresponding to protein-conjugated tetrahedra is also visible. The mobility of the conjugate band varies approximately sinusoidally with attachment position: the mobility difference between the conjugate and the unmodified tetrahedron is maximal for attachment at base 13, and minimal for attachment at base 8 (in this lane the conjugate band is not separated from the unconjugated control). The mobilities of the conjugate bands for attachment at bases 5 and 15, which are separated by one turn of the double helix, are approximately equal. The width of the band is greater for intermediate mobilities than at the extremes. This pattern is consistent with the expected helical trajectory of the attachment point along the edge. We conclude that when the protein is attached at base 13 it is positioned outside the tetrahedron where it decreases the electrophoretic mobility of the construct; when it is attached at base 8 (five bases further along the helix, corresponding to ˜180° rotation) it has minimal effect on mobility because it is contained within the tetrahedral cage.

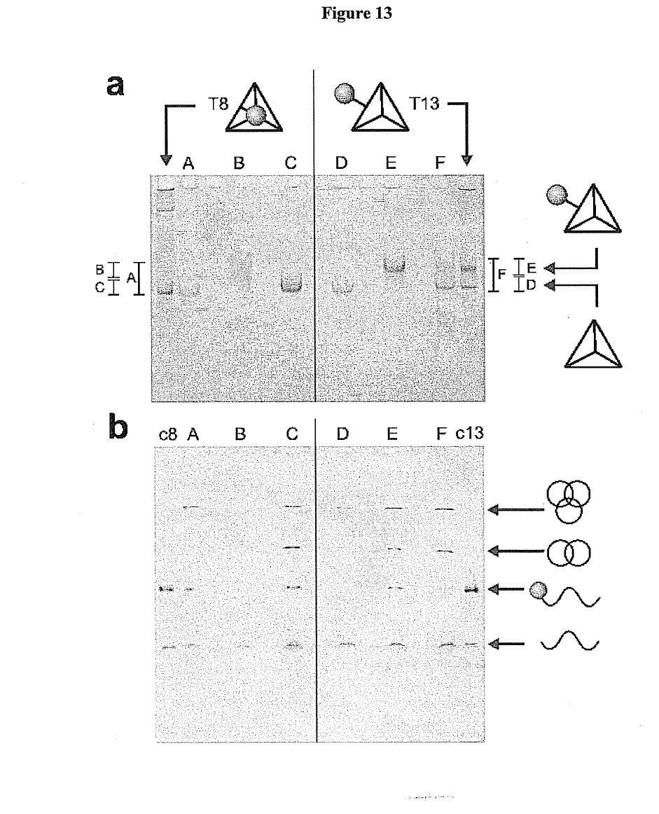

We have analyzed the contents of bands in FIG. 11c to confirm this interpretation. The outer lanes in FIG. 13a, labelled T8 and T13, contain tetrahedra prepared with cytochrome c attached at the 8^{th} and 13^{th} bases, i.e. ‘inside’ and ‘outside’ respectively. These differ from the corresponding lanes in FIG. 11c only in that each of the unmodified oligonucleotides s2-s4 had been ligated after tetrahedral assembly to form linked circles. Oligonucleotides s2-s4 to be ligated were purchased with a 5′ phosphate modification (Integrated DNA Technologies) and ligated by incubating overnight with T4 DNA Ligase (New England Biolabs) at room temperature. For each of these constructs three bands, indicated by the labels A-F, were gel purified in separate gels. Samples A-F were run beside the original constructs in lanes A-F in a native gel (FIG. 13a) and in a denaturing gel in which the hybridization interactions holding the tetrahedra together were disrupted (FIG. 13b). The native gel contained 8% acrylamide (29:1 acrylamide bisacrylamide ratio) and was run in TAE buffer. For the Urea-SDS denaturing gel, samples were combined 1:1 with a loading buffer containing 7M Urea and 3% SDS, held at 95 degrees for 5 minutes, then run on a denaturing gel containing 2.5M Urea and 0.1% SDS with stacking and separating layers containing 5% and 12% acrylamide respectively.

Outer control lanes in FIG. 13b contain the protein-s1 conjugate. As expected, little DNA was found in lane B and all other lanes contained the expected fragments of tetrahedra: three linked circles; two linked circles (a result of a single failed ligation); and single unmodified linear oligonucleotides (unligated s2-s4 or unmodified s1). Of the two bands that had the same mobility in a native gel as an unmodified tetrahedron, C and D, DNA-conjugated cytochrome c was present in band C from T8 and not band D from T13. This is consistent with our interpretation that tetrahedra T8 enclosed cytochrome c whereas the corresponding band from T13 contained only unmodified tetrahedra. The slower band E from T13, which is not present in T8, also contained cytochrome c, consistent with the interpretation that tetrahedra T13 had the protein attached on the outside.

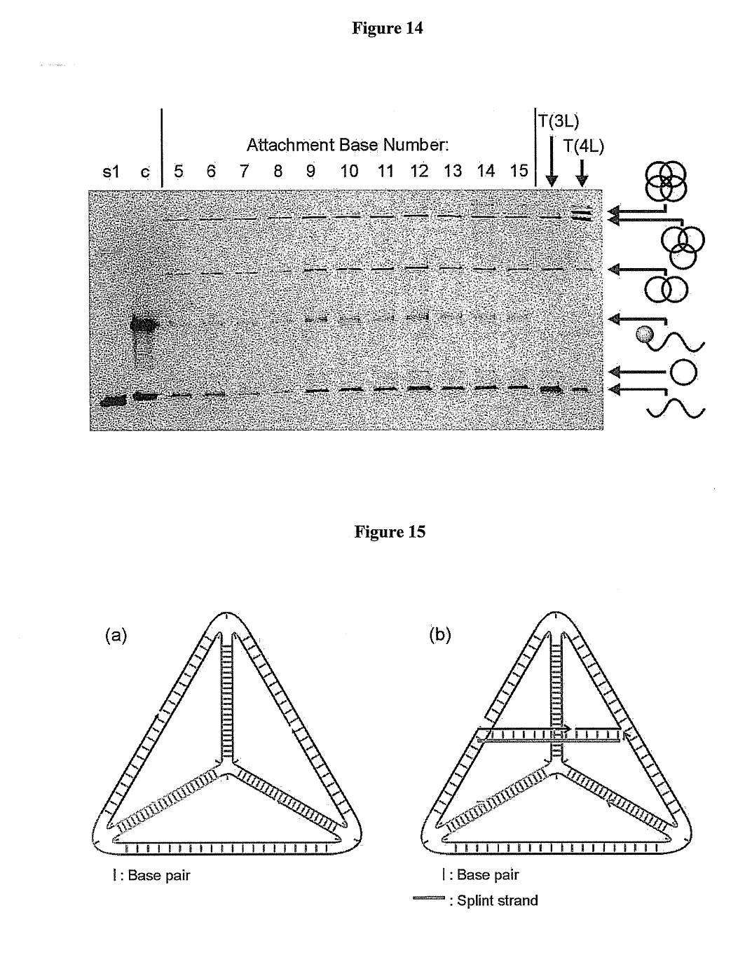

Further denaturing PAGE analysis confirmed the presence of cytochrome c in all bands in FIG. 13c identified as tetrahedron-protein conjugates (FIG. 14).

### Example 5

### Forming crossbridges on the DNA Tetrahedron

In Example 4, we have demonstrated that we can encapsulate a protein inside a DNA tetrahedron cage. Our experiments so far suggest however that the caged protein is still accessible to certain molecules in the environment; in particular an antibody to the protein cytochrome c was shown to bind the caged protein in gel shift experiments. We believe that this is due to the fact that the open faces of the tetrahedral cage provide too much access to the inside of the cage.

We have hence designed modifications to the original self-assembled DNA tetrahedron. The altered design includes a DNA cross-bridge across each of the four tetrahedral faces. With the faces covered up in this way, we believe that access from the environment to the caged molecule will be restricted. FIG. 15 schematically illustrates the design. The cross-bridge is formed by connecting the 3′ end of one strand (left-hand edge) and the 5′ end of another strand (right-hand edge). This cross-bridge is formed by using longer versions of the normal tetrahedra oligos with overhangs on the 5′ and/or 3′ end.

To complete the cross-bridge, the 3′ end of the black strand is ligated to the 5′ end of the red strand. This could be done in two ways. Either the tetrahedra are formed first, a splint strand is added to hold the overhangs in place and then the strands are ligated, for example by DNA Ligase; or the two strands can be covalently linked first (by ligation or in synthesis) before forming the tetrahedra, the linked product is gel-purified on a Urea denaturing gel and tetrahedra are then formed from the linked product and the two other strands.

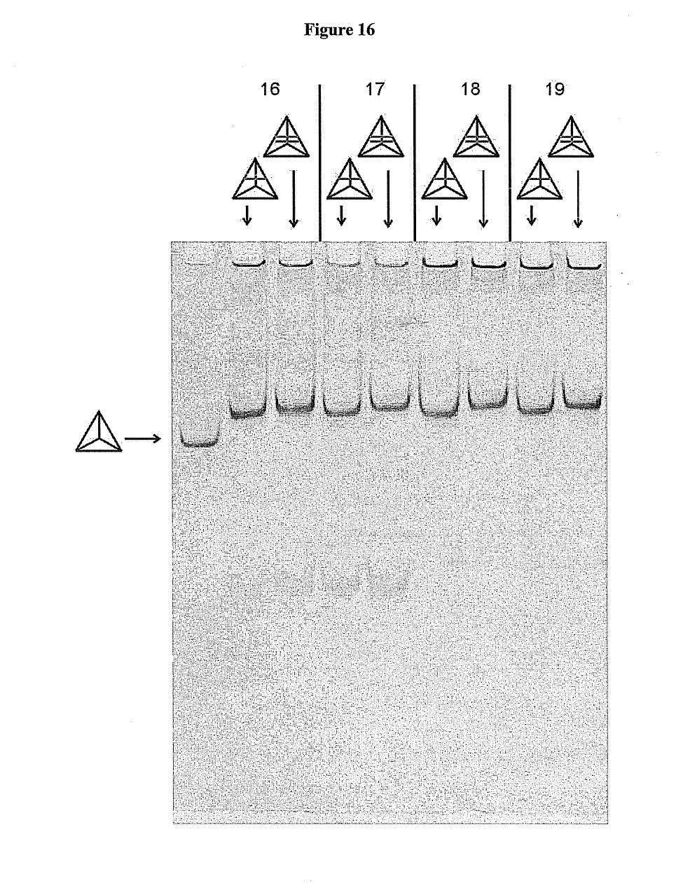

We have so far succeeded in forming tetrahedra with a cross-bridge across one face using the latter approach. The results are shown in the gel in FIG. 16. The left hand lane shows a regular DNA tetrahedron formed from four oligos of equal length. The following lanes show tetrahedra that were formed after ligating two of the strands first and they hence have a cross-bridge across one face. The gel shows tetrahedra with the cross-bridge length varying from 16 to 19 bases. The splint strand can be bound to the single stranded cross-bridge; binding of the splint strand can be seen to cause a small gel shift of the tetrahedra. These experiments indicate that we can form tetrahedra with a bridge across at least one face.

### Example 6

### Opening the Tetrahedron Cage

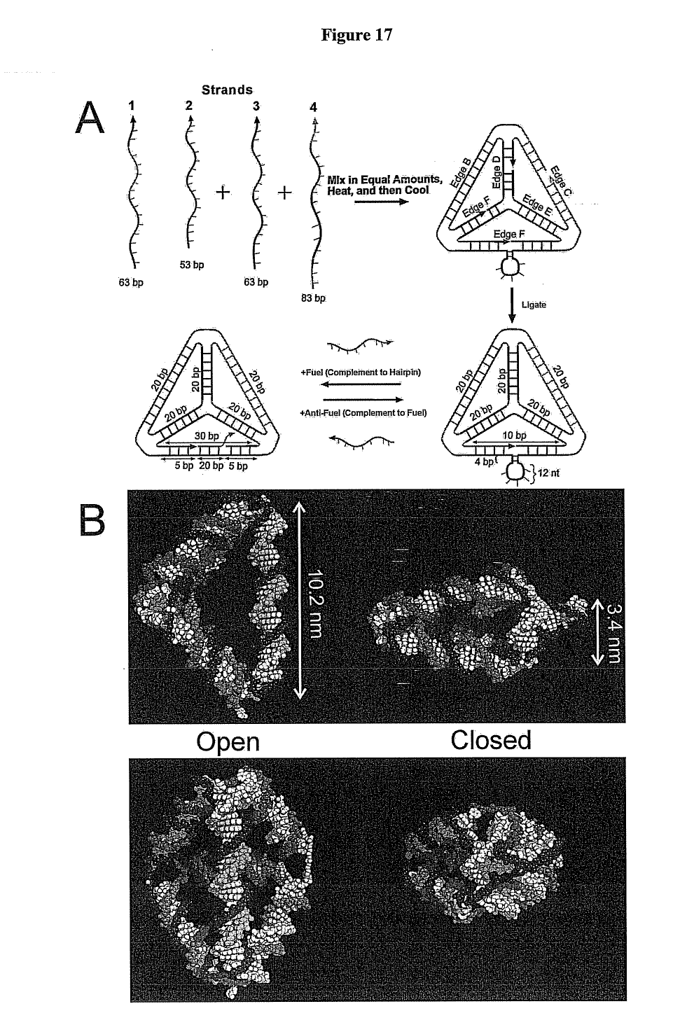

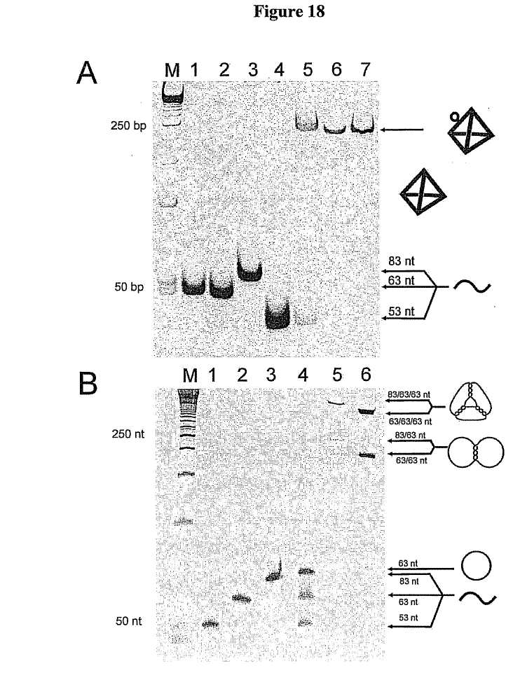

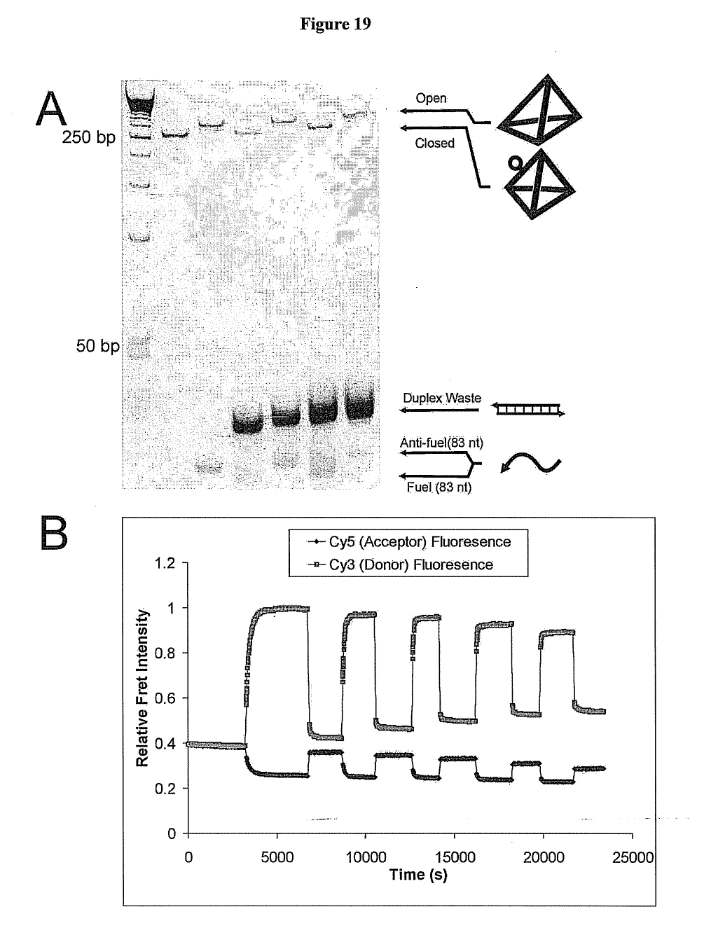

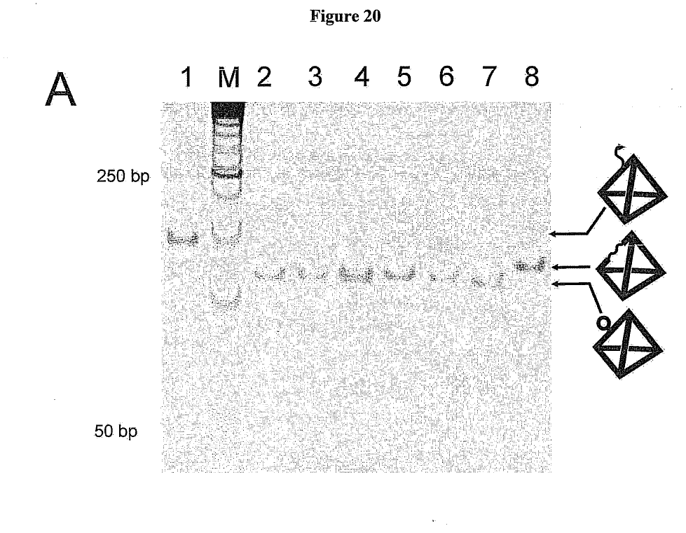

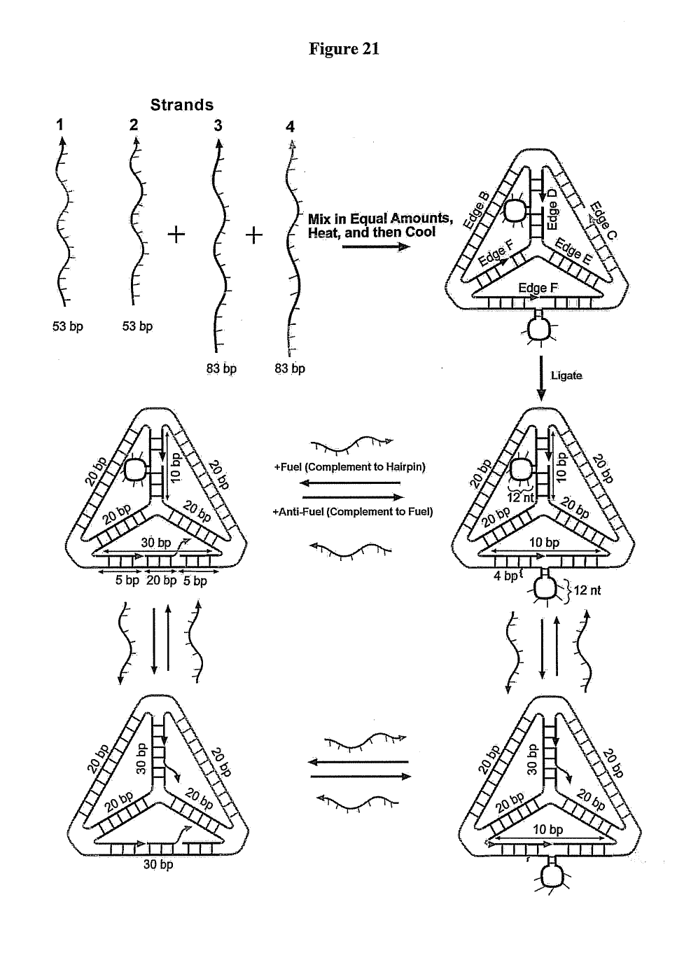

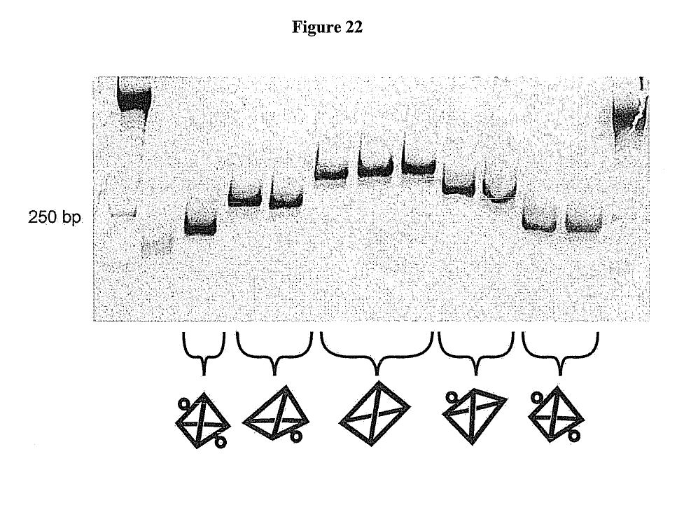

We introduce a simple modification to the design of braced three-dimensional rigid nanostructures, DNA tetrahedra, that allows their edges to be reconfigured in length. By incorporating a hairpin loop into any edge of a DNA tetrahedra, the effective length of that edge may be expanded and contracted at will by the addition of appropriately designed “fuel” and “antifuel” strands. The results are intrinsically three dimensional objects, structurally braced, that may be dynamically modified in dimensions and volume. The results are shown in FIGS. 17 to 22.

### Example 7

### Reconfigurable, Braced, Three-Dimensional DNA Nanostructures

In this Example, we report the incorporation of simple hairpin-loop motifs into one or more edges of a DNA tetrahedron that enable such dynamic capabilities. By the addition of the appropriate complementary DNA strands, the length of the modified edges may be extended or contracted as desired. These conformational changes have been confirmed by both bulk and single-molecule FRET.

Unlike other dynamic DNA devices, DNA tetrahedra are natural building blocks for larger structures: they may be synthesized rapidly and in high-yield, they are stereopure, their triangulated architecture conveys structural stability, and they may be easily joined together. Reconfigurable tetrahedra are thus ideal components for larger, multi-jointed nanostructures, and may also find applications as reconfigurable drug delivery vehicles.

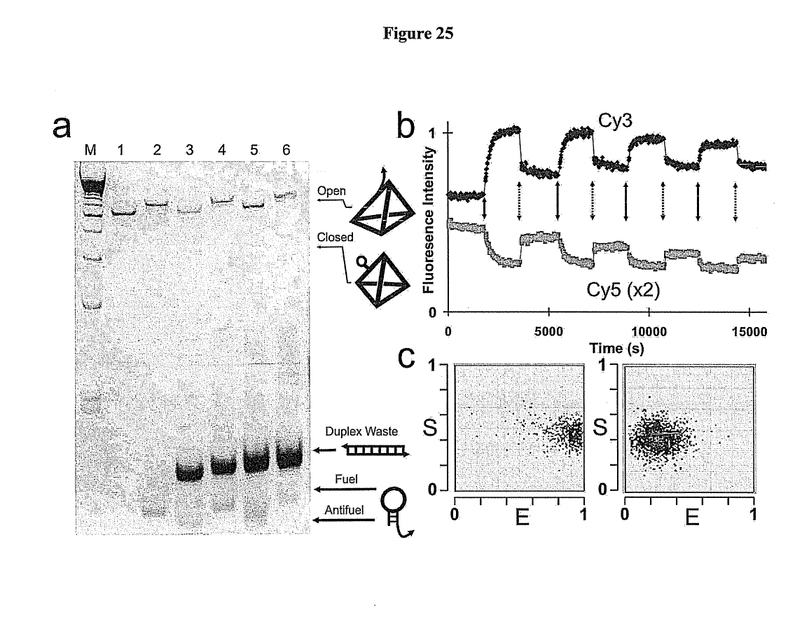

All DNA sequences were designed using NANEV and purchased from Integrated DNA Technologies (IDT). The dual labelled Cy3/Cy5 strand used in FIG. 25 was purchased with dual-HPLC purification; the remaining strands were purchased with PAGE purification.

The sequences of all DNA oligonucleotides are listed in the 5′ to 3′ direction. The presence of a 5′ phosphorylation is indicated by ‘Phos’, and the presence of Cy3 and Cy5 dyes by ‘Cy3’ and ‘Cy5’ respectively.

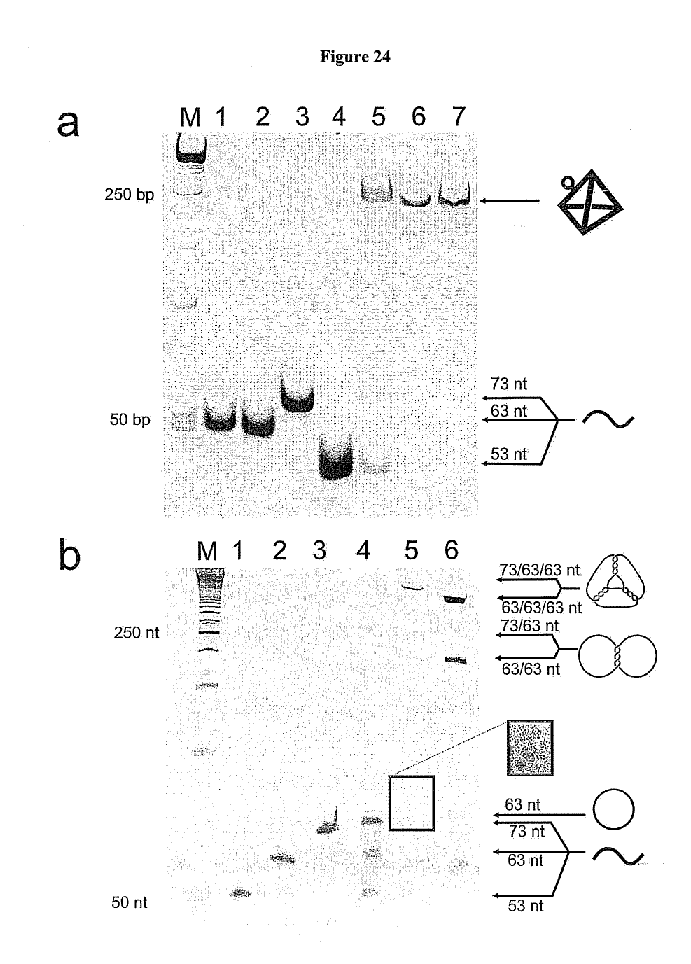

Tetrahedron with a Single Reconfigurable Edge (Gel and FRET Measurements Illustrated in FIG. 25 and FIG. 24).

Strand 1:

(SEQ ID NO: 22)

\Phos\AGGCAGTTGAGACGAACATTCCTAAGTCTGAAATTTATCACCCG
CCATAGTAGACGTATCACC
Strand 2:

(SEQ ID NO: 23)

\Phos\CTTGCTACACGATTCAGACTTAGGAATGTTCGACATGCGAGGGT
CCAATACCGACGATTACAG
Strand 3:

(SEQ ID NO: 24)

\Phos\GGTGATAAAACGTGTAGCAAGCTGTAATCGACTCTAGCGGAAGA
ACCCACAACCGCGGCTCACTACTATGGCG
Strand 4:

(SEQ ID NO: 25)

TAGAGACGGTATTGGACCCTCGCATGACTCAACTGCCTGGTGATACGAGA
GCC
Strand 4(FRET):

(SEQ ID NO: 26)

TAGAGACGGTATTGGACCCTCGCATGACTCAACTGCCTGGTGATACGAGA
GCC
Fuel 1:

(SEQ ID NO: 27)

GCGGTTGTGGGTTCTTCCGCCTTCCTCTCG
Antifuel 1:

(SEQ ID NO: 28)

CGAGAGGAAGGCGGAAGAACCCACAACCGC

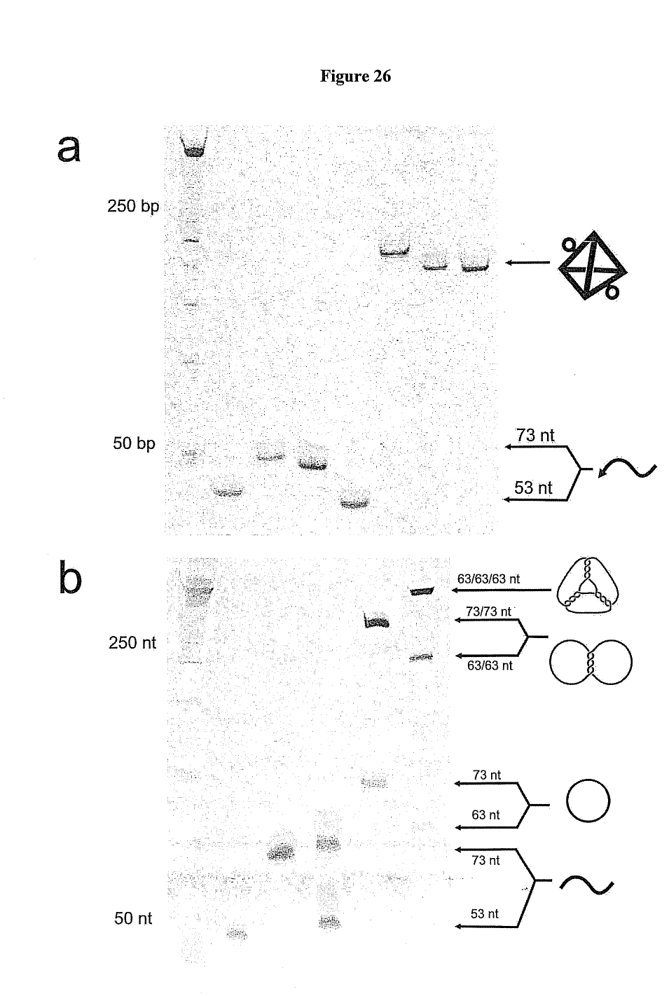

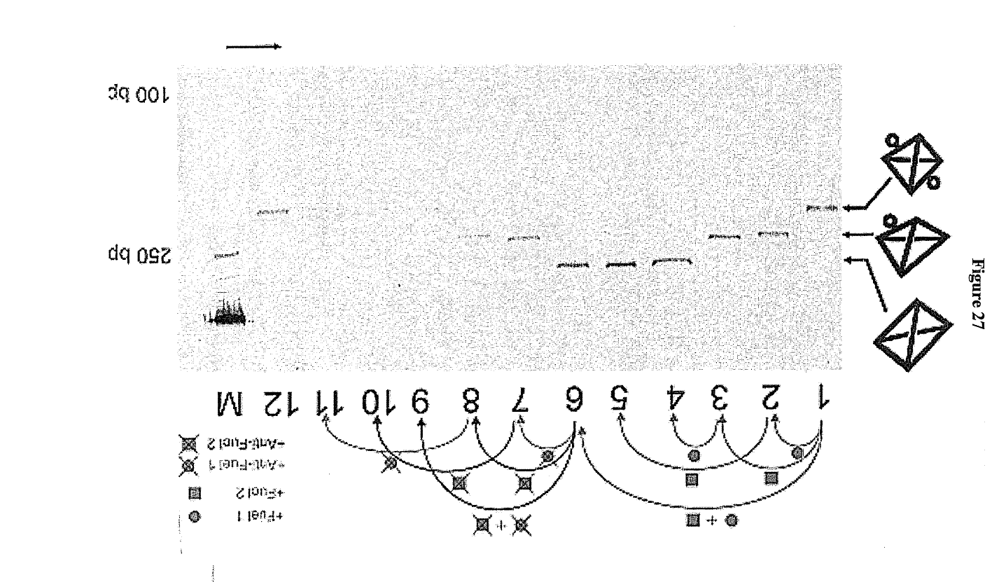

Tetrahedron with a Single Reconfigurable Edge (Gel and FRET Measurements Illustrated in FIG. 27 and FIG. 26).

Strand 1:

(SEQ ID NO: 29)

CTGAAATTTATCACCCGCCATAGTAGACGTATCACCAGGCAGTTGAGACG
AAC
Strand 2:

(SEQ ID NO: 30)

CTTGCTACACGATTCAGGGCGCGAGACCTACAGCGCCGTTCGACATGCGA
GGGTCCAATACCGACGATTACAG
Strand 3:

(SEQ ID NO: 31)

\Phos\GGTGATAAAACGTGTAGCAAGCTGTAATCGACTCTAGCGGAAGA
ACCCACAACCGCGGCTCACTACTATGGCG
Strand 4:

(SEQ ID NO: 32)

TAGAGACGGTATTGGACCCTCGCATGACTCAACTGCCTGGTGATACGAGA
GCC
Fuel 1:

(SEQ ID NO: 33)

GCGGTTGTGGGTTCTTCCGCCTTCCTCTCG
Antifuel 1:

(SEQ ID NO: 34)

CGAGAGGAAGGCGGAAGAACCCACAACCGC
Fuel 2:

(SEQ ID NO: 35)

GGCGTTGTAGGTCTCTCGCCTTCATGGACT
Antifuel 2:

(SEQ ID NO: 36)

AGTCCATGAAGGCGAGAGACCTACAACGCC

Unless otherwise stated, all tetrahedra were formed by combining equal volumes of 0.5 μM solutions of each strand in a buffer containing 15 mM MgCl₂ and 10 mM tris (pH 8), heating to 95° C. for 2 minutes, and then letting cool to room temperature.

T4 DNA ligase and exonuclease III were purchased from New England Biolabs. One μL of enzyme was used for each 100 μL of tetrahedron solution. Ligations were performed overnight a room temperature, and digestions at 37° C. for 3 hours. T4 DNA ligase was removed through gel purification; exonuclease III by phenol/chloroform extraction.

To cycle between the open and closed states of a given edge, the appropriate fuel or anti-fuel strand was added to the solution, the solution was heated to 37° C. for 10 minutes, and then let cool to room temperature.

Samples were gel purified by running the appropriate sample on a 6% 19:1 PAGE gel in 1×TAE, staining with SYBR Gold (Molecular Probes), and excising the band. The band was then ground into a fine powder to which 3 volumes of the appropriate buffer was added to elute the desired band. After soaking overnight at room temperature, the residual gel powder was filtered off using a 0.2 μm filtration spin column. The sample was concentrated as desired by using microcon YM-10 centrifugation column.

FRET experiments were conducted using a JY-Horiba Fluoromax 3 spectrofluorimeter. A water bath was used to maintain the sample at a specified temperature (37° C.).

FRET experiments were conducted in a total volume of 1.5 mL, using 100 uL of sample and an additional 1.4 mL of buffer in a quartz cuvette (Hellma UK Ltd, UK). At each point of addition of DNA, the sample was mixed by rapid pipetting action for 10 seconds. An approximately 10 nM final concentration of gel-purified, fluorescently labelled tetrahedron was used. Each addition of fuel or anti-fuel used a 10% stoichiometric excess of that of the previous addition.

Donor excitation was at 550 nm, and donor emission was measured at 565 nm. Acceptor emission measured at 665 nm.

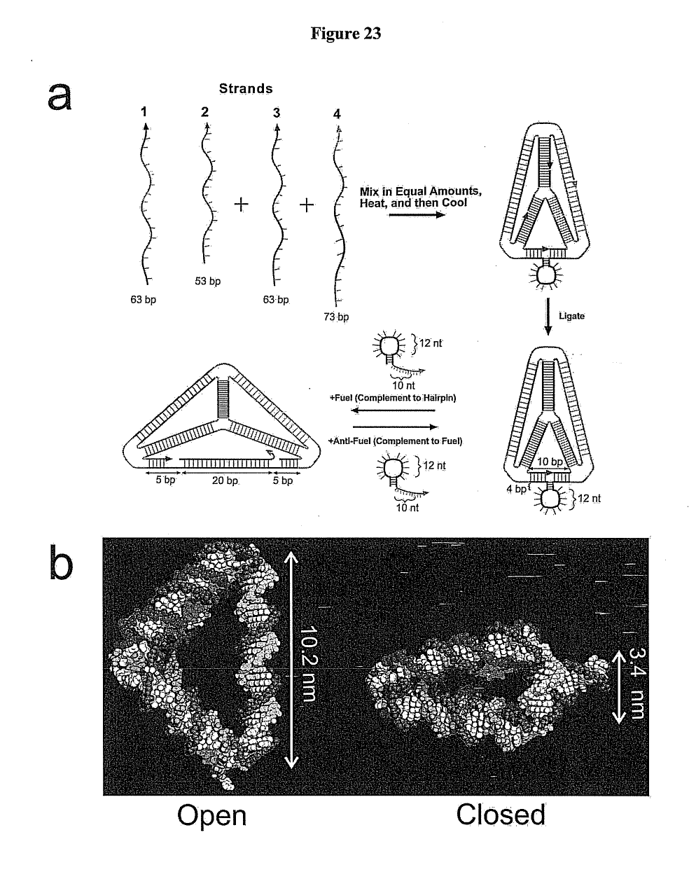

FIG. 23a illustrates the synthesis scheme of a DNA tetrahedron containing a single reconfigurable edge. As with the previously reported DNA tetrahedra, four short DNA strands are designed to contain six sets of complementary subsequences that, when combined in solution, produce a DNA construct with tetrahedral connectivity. Each set of complementary subsequences hybridize to form a single edge, and each strand runs round once face. The scheme has been modified at one edge to include a DNA hairpin loop in one strand, opposite a nick in the complementary strand. In this specific example, the tetrahedron possesses five edges that are 20 bp (6.8 nm) in length, while the remaining edge is approximately 10 bp (3.4 nm) in length and contains a hairpin loop with a four basepair neck and a 12 nucleotide loop region.

This hairpin loop is the basis for the reconfigurability of the tetrahedron: when the hairpin is formed, the edge is in its ‘closed’ state and possesses its shortest possible length. In this state, the two five-bp sections of the edge and the hairpin form a nicked, three-way junction. In its ‘open’ state, the entire length of the hairpin is hybridized to a ‘fuel’ strand. The result is a continuous duplex edge with two nicks, positioned at the ends of the hairpin section of the arm. The full length of this edge is expected to be 10.2 nm, the length of a 30 bp duplex edge. FIG. 23b illustrates a space filling representation of both the open and closed states of the tetrahedron.

To switch from the closed state to the open state, the addition of only the fuel strand—fully complementary to the hairpin loop—is required. The large energetic gain from the formation of Watson-Crick basepairs in the neck of the hairpin loops drives this hybridization and the resulting change in dimensions of the tetrahedron. To enable the switch from the open state to the closed state an extensions of the fuel strand, designed to remain unhybridized in the open state, is incorporated. When a strand fully complementary to the fuel and its unhybridized extension strand (an ‘antifuel’ strand) is added, the fuel strand is displaced from the tetrahedron, returning it to the original closed state. It should be noted that, as the fuel strand is complementary to the hairpin loop of the tetrahedron, it is itself a hairpin. So to is the anti-fuel strand. This scheme represents perhaps the simplest possible motif for a edge contraction/extension in such a structure: A change in conformation may be accomplished in a single step with the addition of a single strand. The resulting conformational change is great: In the example illustrated in FIG. 23 the edge's length increases by a factor of approximately 3 times upon extension.

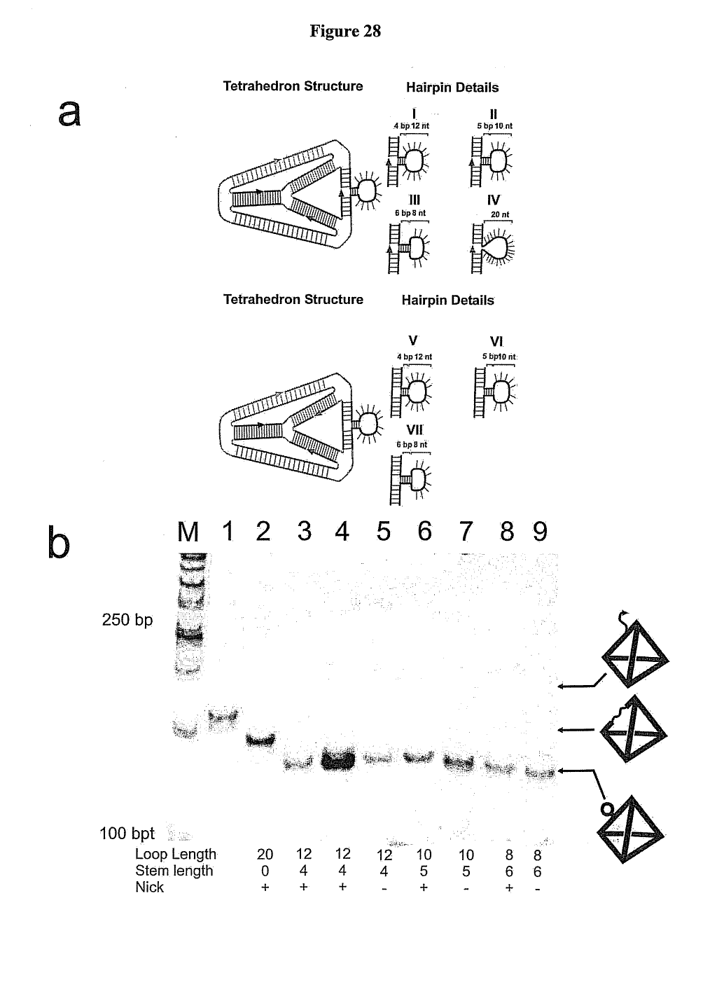

FIG. 24 illustrates the formation of a reconfigurable DNA tetrahedron with dimensions as illustrated in FIG. 23. As with previously demonstrated syntheses, the tetrahedron may be formed in a matter of minutes and in high (>95%) yield, and ligation of strands produces exactly the expected circular catenanes on a denaturing gel, confirming the structure is as designed. A number of control experiments confirm that the hairpin loop is properly formed at room temperature in 15 mM MgCl₂ (see FIG. 28).

FIG. 25 demonstrates the extension and contraction of the hairpin-loop-containing edge. In FIG. 25a, the sequential addition of fuel and antifuel strands in stoichiometric excess (to illustrate waste products) produces exactly the expected mobility shifts. The tetrahedron in its closed state (lane 1), when combined with a fuel strand (evident in the higher mobility areas of the gel), is converted into a lower mobility product as a result of its increased dimensions (lane 2). When excess antifuel is added, the original tetrahedron band is restored, and antifuel and waste duplex product are observed (lane 3). Repeated cycles of opening and closing increase produce the same effect (lanes 4-6).

FIG. 25b illustrates a bulk FRET investigation into the conformation changes, carried out with a tetrahedron modified with Cy3 and Cy5 dyes at the 5′ and 3′ positions of the nick opposite at the hairpin. In the closed state of the tetrahedron, the close proximity of the dyes are expected to result in a relatively low Cy3 fluorescence and a high Cy5 fluorescence; in the open state, the Cy3 fluorescence is expected to increase, and the Cy5 fluorescence decrease. The addition of the fuel strand (indicated by the green arrows) and anti-fuel strands (indicated by the black arrows) produce exactly the expected changes in fluorescence intensity, and offer further confirmation of the expected structural changes. It is interesting to note the decay in the magnitude of the fluorescence changes as the cycling progresses. A similar effect is observed in FRET experiments with dynamic DNA devices; it is possible the build-up of waste duplex product might interfere in some way with the operation of the device. FIG. 25c illustrates single molecule FRE5 measurements of the conversion of a population of closed tetrahedra to open tetrahedra. The conversion between the states is near 100%, and the distribution illustrates only a single population in each state.

Given the simplicity of the incorporation of a single hairpin-loop into a tetrahedron edge, the incorporation of two hairpin loops on opposite edges of a tetrahedron was next investigated. FIG. 25 illustrates native and denaturing gel analysis of a construct containing two such modified opposite edges, each containing a similar hairpin with a different sequence (and thus independently addressable). As with all other previously reported tetrahedra, the tetrahedron may be formed in rapidly, in a single step and in high yield. Denaturing gel analysis of the ligated product reveals exactly the expected circular catenanes (FIG. 26).

With two independently addressable hairpin loops, a tetrahedron with two reconfigurable edges possesses four different states. Each of these states is illustrated in FIG. 27, and a number of different transitions from one state to another are illustrated. For example, the tetrahedron may be converted from the entirely closed to the entirely open state in a number of ways; through the opening of one hairpin (lanes 2 and 3) followed by the other (lanes 4 and 5), or through the simultaneous opening of both hairpins. Each hairpin may be closed in a similar manner.

1. A nucleic acid polyhedron having a moiety contained therein.

2. A nucleic polyhedron according to claim 1, wherein the nucleic acid is DNA.

3. A nucleic acid polyhedron according to claim 1, wherein the polyhedron is a tetrahedron and/or the polyhedron comprises at least one nucleic acid cross-bridge.

4. (canceled)

5. A nucleic acid polyhedron according to claim 1, wherein the structure of the polyhedron has been modified for release of or for access to the moiety.

6. A nucleic acid polyhedron according to claim 1, wherein the moiety is a protein, optionally cytochrome c, or nucleic acid.

7. (canceled)

8. A method of making a nucleic acid polyhedron having a moiety contained therein, comprising:
(a) providing one or more oligonucleotides, each oligonucleotide comprising subsequences that hybridize to subsequences on the same oligonucleotide or a different oligonucleotide to allow the oligonucleotide(s) to form the polyhedron;
(b) mixing the oligonucleotide(s) of (a) and the moiety in a buffer solution; and
(c) optionally subjecting the mixture of (b) to a heat treatment comprising a heating step to denature the oligonucleotide(s) and a cooling step to anneal the oligonucleotide(s) to allow them to hybridize to form thereby a nucleic acid polyhedron;
wherein step (b) and step (c) if it is performed are carried out in such a manner that the moiety becomes contained within the nucleic acid polyhedron.

9. A method according to claim 8, wherein the nucleic acid is DNA.

10. A method according to claim 8, wherein the subsequences have linkers therebetween.

11. A method according to claim 10, wherein the linkers comprise nucleotides.

12. A method according to claim 8, wherein the moiety is attached to one of the oligonucleotides before step (b).

13. A method according to claim 8, wherein step (b) and step (c) if it is performed are carried out in the presence of lipids which cause the moiety to be contained inside the nucleic acid polyhedron.

14. A method according to claim 8, wherein the polyhedron is a tetrahedron.

15. A method according to claim 8, wherein different oligonucleotides comprise subsequences having linkers therebetween to allow each oligonucleotide to form the edge of each face of the polyhedron and wherein the subsequences are selected such that each subsequence hybridizes to the subsequence of the oligonucleotides on the shared edges of the adjacent polyhedron faces.

16. A method according to claim 15, wherein four oligonucleotides are provided and each oligonucleotide comprises three different subsequences having linkers there between, wherein each first subsequence is capable of hybridizing to the first subsequence of one of the other three oligonucleotides, each second subsequence is capable of hybridizing to the second subsequence of another of the three oligonucleotides and each third subsequence is capable of hybridizing to the third subsequence of the third of the other oligonucleotides.

17. A method according to claim 16 wherein:
the first subsequence of the first oligonucleotide is capable of hybridizing to the first subsequence of the third oligonucleotide;
the second subsequence of the first oligonucleotide is capable of hybridizing to the second subsequence of the second oligonucleotide;
the third subsequence of the first oligonucleotide is capable of hybridizing to the third subsequence of the fourth oligonucleotide;
the first subsequence of the second oligonucleotide is capable of hybridizing to the first subsequence of the fourth oligonucleotide;
the third subsequence of the second oligonucleotide is capable of hybridizing to the third subsequence of the third oligonucleotide; and
the second subsequence of the third oligonucleotide is capable of hybridizing to the second subsequence of the fourth oligonucleotide.

18. A method according to claim 14, wherein four oligonucleotides are provided and each oligonucleotide comprises four different subsequences having linkers therebetween and wherein:
the first and fourth subsequence of the first oligonucleotide are capable of hybridizing to the second subsequence of the fourth oligonucleotide;
the first and fourth subsequence of the second oligonucleotide are capable of hybridizing to the second subsequence of the third oligonucleotide;
the first and fourth subsequence of the third oligonucleotide are capable of hybridizing to the third subsequence of the first oligonucleotide;
the first and fourth subsequence of the fourth oligonucleotide are capable of hybridizing to the third subsequence of the second oligonucleotide;
the second subsequence of the first oligonucleotide is capable of hybridizing to the second subsequence of the second oligonucleotide; and
the third subsequence of the third oligonucleotide is capable of hybridizing to the third subsequence of the fourth oligonucleotide.

19. A method according to claim 10, wherein the length of each subsequence and linker is selected to form a regular tetrahedron.

20. A method according to claim 8, wherein the buffer comprises counter ions such as Na+, Ca2+ or Mg2+.

21. A method according to claim 8, wherein the method further comprises (a) forming at least one nucleic acid cross-bridge and/or (b) modifying the stricture of the polyhedron to allow for release of or for control of access to the moiety in response to a trigger or synthesis.

22. (canceled)

23. A nucleic acid polyhedron having a moiety attached to the outside of the polyhedron.
